(19) United States
(12) Patent Application Publication
(10) Pub. No.: US 2025/0339502 A1
(43) Pub. Date: Nov. 6, 2025

(54) RIBONUCLEASES FOR TREATING VIRAL INFECTIONS

(71) Applicant: Orgenesis Inc., Germantown, MD (US)

(72) Inventors: JAMIE SULLEY, La Jolla, CA (US); LUIS SQUIQUERA, Buenos Aires (AR); THOMAS HODGE, Athens, GA (US); SABINA GLOZMAN, Naharya (IL)

(73) Assignee: Orgenesis Inc., Germantown, MD (US)

(21) Appl. No.: 17/906,631

(22) PCT Filed: Mar. 19, 2021
(86) PCT No.: PCT/US2021/023276

§ 371 Date: Sep. 19, 2022

Related U.S. Application Data
(60) Provisional application No. 62/992,408, filed on Mar. 20, 2020.
(60) Provisional application No. 63/005,582, filed on Apr. 6, 2020.
(60) Provisional application No. 63/010,045, filed on Apr. 14, 2020.
(60) Provisional application No. 63/040,007, filed on Jun. 17, 2020.

(51) Int. Cl. A61K 38/51; A61K 31/49; A61K 35/15; A61K 35/17; A61P 31/14; C07K 16/10

(52) U.S. Cl. CPC A61K 38/51; A61K 31/49; A61K 35/15; A61K 35/17; A61P 31/14; C07K 16/1003; C12Y 406/01

(57) ABSTRACT

This disclosure is directed to compounds and pharmaceutical compositions for treating and preventing viral diseases, as Covid-19. Among others, the invention relates to the use of immune cells and ribonucleases in the preparation and use of pharmaceutical formulations for the treatment of said disease.

### FIELD OF THE DISCLOSURE

This disclosure is directed to compounds and pharmaceutical compositions for treating and preventing viral diseases, as Covid-19. Among others, the invention relates to the use of immune cells and ribonucleases in the preparation and use of pharmaceutical formulations for the treatment of said disease.

### SEQUENCE LISTING STATEMENT

The instant application contains a Sequence Listing which has been submitted electronically in ASCII format and is hereby incorporated by reference in its entirety. Said ASCII copy, created on Apr. 27, 2023, is named P-603948-US-SQL_ST25 and is 5,378 bytes in size.

### BACKGROUND

Several human diseases are caused by viruses, as the common cold, influenza, chickenpox, cold sores, rabies, Ebola virus disease, AIDS (HIV), avian influenza, SARS, and Covid-19. These diseases are usually detected by clinical presentation, for instance severe muscle and joint pains preceding fever, or skin rash and swollen lymph glands.

Coronaviruses (CoV) are a large family of viruses that cause illness ranging from the common cold to more severe diseases such as Middle East Respiratory Syndrome (MERS-CoV), Severe Acute Respiratory Syndrome (SARS-CoV), and Covid-19. Coronaviruses are in the subfamily Orthocoronavirinae in the family Coronaviridae, in the order Nidovirales. They are enveloped viruses with a positive-sense single-stranded RNA genome and a nucleocapsid of helical symmetry. The genome size of coronaviruses ranges from approximately 26 to 32 kilobases, the largest for an RNA virus. Coronaviruses are zoonotic, meaning they are transmitted between animals and people. Coronaviruses further cause colds with major symptoms, such as fever and sore throat from swollen adenoids, primarily in the winter and early spring seasons, pneumonia, and bronchitis, among others.

The novel coronavirus SARS-CoV-2, informally known as the Wuhan coronavirus, is a contagious virus that causes acute respiratory diseases, and has been the cause of a major virus outbreak known as 2019-20 Wuhan coronavirus outbreak. The virus is thought to have a zoonotic origin, as suggested by its similarity to SARS-CoV and bat coronaviruses. However, human-to-human transmission of the virus has been confirmed, primarily through close contact, in particular through respiratory droplets from coughs and sneezes. Viral RNA has also been found in stool samples from infected patients

There are no vaccines or antiviral drugs to prevent or treat human coronavirus infections.

### SUMMARY OF THE INVENTION

In one aspect, disclosed herein is a composition for treating or preventing a viral disease in a subject, said composition comprising a ribonuclease. In some related aspects, the ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

In some related aspects, the ribonuclease comprises ranpirnase. In some related aspects, the viral disease is caused by a virus selected from a group comprising severe acute respiratory syndrome coronavirus 2 (SARS-CoV-2), an adenovirus, a herpesvirus, a papillomavirus, a polyomavirus, a poxvirus, an hepadnavirus, a parvovirus, an astrovirus, a calicivirus, a picornavirus, a coronavirus, a flavivirus, a togavirus, a hepevirus, a retrovirus, an orthomyxovirus, an arenavirus, a bunyavirus, a filovirus, a paramyxovirus, a rhabdovirus, a reovirus, Herpes simplex type 1, Herpes simplex type 2, Varicella-zoster virus, Epstein-Barr virus, Human cytomegalovirus, human herpesvirus type 8, human papillomavirus, BK virus, JC virus, smallpox, Hepatitis B virus, parvovirus B19, human astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, Banna virus, or any combination thereof.

In some related aspects, the viral disease is selected from a group comprising acute hepatitis, AIDS, aseptic meningitis, bronchiolitis, Burkitt's lymphoma, chickenpox, chronic hepatitis, common cold, congenital rubella, congenital varicella syndrome, congenital seizures in the newborn, croup, cystitis, cytomegalic inclusion disease, fatal encephalitis, gastroenteritis, German measles, gingivostomatitis, hepatic cirrhosis, hepatocellular carcinoma, herpes labialis, cold sores, herpes zoster, Hodgkin's lymphoma, hyperplastic epithelial lesions, warts, laryngeal papillomas, epidermodysplasia verruciformis, infectious mononucleosis, influenza, influenza-like syndrome, Kaposi sarcoma, keratoconjunctivitis, liver, lung and spleen diseases in the newborn, malignancies, cervical carcinoma, squamous cell carcinomas, measles, multicentric Castleman disease, mumps, myocarditis, nasopharyngeal carcinoma, pericarditis, pharyngitis, pharyngoconjunctival fever, pleurodynia, pneumonia, poliomyelitis, postinfectious encephalomyelitis, premature delivery, primary effusion lymphoma, rabies, Reye syndrome, severe bronchiolitis with pneumonia, skin vesicles, mucosal ulcers, tonsillitis, pharyngitis, or combination thereof.

In some related aspects, the viral disease comprises Covid-19, or wherein said viral disease is caused by severe acute respiratory syndrome coronavirus 2 (SARS-CoV-2). In some related aspects, the composition further comprises quinine, chloroquine, hydroxychloroquine, or analogues or derivatives thereof. In some related aspects, the composition further comprises an angiotensin-converting enzyme inhibitor (ACE-I). In some related aspects, the composition further comprises immunoglobulins, fragments thereof, antibodies, or combinations thereof, obtained from a plasma of a subject immune to said viral disease.

In some related aspects, the immunoglobulins are IgG, IgM or combinations thereof. In some related aspects, the immunoglobulin fragments are F(ab′)2 fragments. In some related aspects, the viral disease comprises Covid-19, and said plasma is collected from healthy subject or pool of subjects who have been previously exposed to SARS-CoV-2, naturally or by deliberate immunization, and who have IgG or IgM antibodies to SARS-CoV-2 virus in their plasma.

In some related aspects, the viral disease comprises Covid-19, and said plasma is collected from a subject or pool of subjects where SARS-CoV-2 infection rate is high. In some related aspects, the viral disease comprises Covid-19, and said plasma is collected from a subject or pool of subjects who have a history of SARS-CoV-2 infection in the past. In some related aspects, the viral disease comprises Covid-19, and said plasma is collected from a subject or pool of subjects who are found to have IgG or IgM antibodies to SARS-CoV-2 through an antibody screening program.

In some related aspects, the viral disease comprises Covid-19, and said plasma is collected from a subject or pool of subjects who have antibodies as the result of deliberate immunization with SARS-CoV-2 or with antigens associated with SARS-CoV-2. In some related aspects, the viral disease comprises Covid-19, and said plasma is collected by either plasmapheresis or after separation from whole blood donations.

In some related aspects, the viral disease comprises Covid-19, and said plasma is collected from:
a. a healthy subject or pool of subjects who have been previously exposed to SARS-CoV-2, naturally or by deliberate immunization, and who have IgG or IgM antibodies to SARS-CoV-2 virus in their plasma; or
b. a subject or pool of subjects where SARS-CoV-2 infection rate is high; or
c. a subject or pool of subjects who have a history of SARS-CoV-2 infection in the past; or
d. a subject or pool of subjects who have antibodies as the result of deliberate immunization with SARS-CoV-2 or with antigens associated with SARS-CoV-2; or
e. any combination thereof.

In some related aspects, the composition further comprises immune cells. In some related aspects, the immune cells are selected from a group comprising neutrophils, eosinophils (acidophiles), basophils, lymphocytes, monocytes, B cells, memory B cell, regulatory B cells (Breg), T cells, cytotoxic T cells, Helper T cells, Th1 cells, Th2 cells, Regulatory T cells (Treg), memory T cells, Natural Killer (NK) cells, monocytes, dendritic cells, macrophages, myeloid dendritic cells (mDC), plasmacytoid dendritic cell (pDC), or a combination thereof.

In some related aspects, the immune cells comprise NK cells. In some related aspects, the immune cells are obtained from a donor, or from a cell line. In some related aspects, the immune cells are obtained from a subject immune to said viral disease.

In some aspects, disclosed herein is a composition comprising a ribonuclease and immunoglobulins, fragments thereof, antibodies, or combinations thereof, obtained from a plasma of a subject immune to a viral disease. In some aspects, disclosed herein is a composition comprising a ribonuclease and immune cells.

### BRIEF DESCRIPTION OF THE DRAWINGS

The subject matter disclosed herein is particularly pointed out and distinctly claimed in the concluding portion of the specification. The compositions and methods for using thereof disclosed herein may best be understood by reference to the following detailed description when read with the accompanying drawings in which:

FIG. 1 shows an assay of ranpirnase and FDA-Approved Drugs efficacy against SARS-CoV-2 in vitro.

FIG. 2 shows an assay of ranpirnase cytotoxicity in vitro.

FIG. 3 shows ranpirnase effect on viral load in vitro.

FIG. 4 shows ranpirnase effect on SARS-CoV-2 number of genomic copies.

### DETAILED DESCRIPTION

In some embodiments, disclosed herein is a composition for treating or preventing a viral disease in a subject, said composition comprising ribonuclease. In some embodiments, disclosed herein is a composition for treating or preventing a Covid-19 in a subject, said composition comprising ribonuclease.

In some embodiments, disclosed herein is a composition for treating or preventing a viral disease in a subject, said composition comprising immunoglobulins, fragments thereof, antibodies, or combinations thereof, obtained from a plasma of a subject immune to said viral disease. In some embodiments, disclosed herein is a composition for preventing Covid-19 in a subject, said composition comprising immunoglobulins, fragments thereof, antibodies, or combinations thereof, obtained from a plasma of a subject immune to Covid-19.

In some embodiments, disclosed herein is a composition for treating or preventing a viral disease in a subject, said composition comprising immune cells. In some embodiments, disclosed herein is a composition for treating or preventing a Covid-19 in a subject, said composition comprising immune cells.

In some embodiments, disclosed herein is a composition for treating or preventing a viral disease in a subject, said composition comprising a ribonuclease and immunoglobulins, fragments thereof, antibodies, or combinations thereof, obtained from a plasma of a subject immune to said viral disease. In some embodiments, disclosed herein is a composition for treating or preventing a Covid-19 in a subject, said composition comprising a ribonuclease and immunoglobulins, fragments thereof, antibodies, or combinations thereof, obtained from a plasma of a subject immune to Covid-19.

In some embodiments, disclosed herein is a composition for treating or preventing a viral disease in a subject, said composition comprising a ribonuclease and immune cells. In some embodiments, disclosed herein is a composition for treating or preventing a Covid-19 in a subject, said composition comprising a ribonuclease and immune cells.

In some embodiments, disclosed herein is a composition for treating or preventing a viral disease in a subject, said composition comprising a ribonuclease, immunoglobulins, fragments thereof, antibodies, or combinations thereof, obtained from a plasma of a subject immune to said viral disease, and immune cells. In some embodiments, disclosed herein is a composition for treating or preventing a Covid-19 in a subject, said composition comprising a ribonuclease, immunoglobulins, fragments thereof, antibodies, or combinations thereof, obtained from a plasma of a subject immune to Covid-19, and immune cells.

In some embodiments, disclosed herein is a composition for treating or preventing a viral disease in a subject, said composition comprising a immunoglobulins, fragments thereof, antibodies, or combinations thereof, obtained from a plasma of a subject immune to said viral disease, and immune cells. In some embodiments, disclosed herein is a composition for treating or preventing a Covid-19 in a subject, said composition comprising immunoglobulins, fragments thereof, antibodies, or combinations thereof, obtained from a plasma of a subject immune to Covid-19, and immune cells.

In some embodiments, disclosed herein is a composition for treating or preventing a viral disease in a subject, said composition comprising immunoglobulins, fragments thereof, antibodies, or combinations thereof, obtained from a plasma of a subject immune to a viral disease, ribonuclease-loaded bioxomes, and immune cells. In some embodiments, disclosed herein is a composition for treating or preventing a Covid-19 in a subject, said composition comprising immunoglobulins, fragments thereof, antibodies, or combinations thereof, obtained from a plasma of a subject immune to a viral disease, ribonuclease-loaded bioxomes, and immune cells.

### Ribonucleases

In some embodiments, disclosed herein is a composition for treating or preventing a viral disease in a subject comprising a ribonuclease. In some embodiments, disclosed herein is a composition for treating or preventing autoimmune disease, malaria, a coronavirus infection, and obesity in a subject comprising a ribonuclease. A skilled artisan would appreciate that ribonucleases, or RNases, are a type of nuclease that catalyzes the degradation of RNA into smaller components. RNases comprise a first defense against RNA viruses, and provide the underlying machinery for more advanced cellular immune strategies such as RNAi. Several types of RNases can be used to degrade RNA, all of them can be used for the compositions and methods disclosed herein.

In some embodiments, a ribonuclease comprises RNase A. In some embodiments, a ribonuclease comprises RNase H. In some embodiments, a ribonuclease comprises RNase III. In some embodiments, a ribonuclease comprises RNase L. In some embodiments, a ribonuclease comprises RNase P. In some embodiments, a ribonuclease comprises RNase PhyM. In some embodiments, a ribonuclease comprises RNase T1.

In some embodiments, a ribonuclease comprises RNase T2. In some embodiments, a ribonuclease comprises RNase U2. In some embodiments, a ribonuclease comprises RNase V. In some embodiments, a ribonuclease comprises PNPase. In some embodiments, a ribonuclease comprises RNase PH. In some embodiments, a ribonuclease comprises RNase R. In some embodiments, a ribonuclease comprises RNase D. In some embodiments, a ribonuclease comprises RNase T.

In some embodiments, a ribonuclease comprises oligoribonuclease. In some embodiments, a ribonuclease comprises exoribonuclease I. In some embodiments, a ribonuclease comprises exoribonuclease II. In some embodiments, a ribonuclease comprises binase. In some embodiments, a ribonuclease comprises MCPIP1. In some embodiments, a ribonuclease comprises eosinophil cationic protein (ECP). In some embodiments, a ribonuclease comprises eosinophil derived neurotoxin (EDN).

In some embodiments, a ribonuclease comprises RNase 3. In some embodiments, a ribonuclease comprises onconase. In some embodiments, a ribonuclease comprises rAmphinase. In some embodiments, a ribonuclease comprises rAmphinase 2. In some embodiments, a ribonuclease comprises bovine seminal RNase (BS_RNase).

In some embodiments, a ribonuclease comprises a human ribonuclease. In some embodiments, a ribonuclease comprises a mammalian ribonuclease. In some embodiments, a comprises a microbial ribonuclease. In some embodiments, a ribonuclease comprises a frog ribonuclease. In some embodiments, a ribonuclease comprises a frog oocytes ribonuclease. In some embodiments, a ribonuclease comprises an artificial ribonuclease. In some embodiments, more than one type of ribonuclease is used in the compositions and methods disclosed herein.

In some embodiments, a ribonuclease degrades tRNA. In some embodiments, a ribonuclease degrades rRNA. In some embodiments, a ribonuclease degrades mRNA. In some embodiments, a ribonuclease is conjugated to a molecule. In some embodiments, a ribonuclease is conjugated to human serum albumin.

In some embodiments, a ribonuclease comprises ranpirnase. In some embodiments, disclosed herein is a composition comprising ranpirnase and immunoglobulins, fragments thereof, antibodies, or combinations thereof, obtained from a plasma of a subject immune to a viral disease. In some embodiments, disclosed herein is a composition comprising ranpirnase and immune cells. In some embodiments, disclosed herein is a composition comprising ranpirnase, immunoglobulins, fragments thereof, antibodies, or combinations thereof, obtained from a plasma of a subject immune to said viral disease, and immune cells. In some embodiments, disclosed herein is a composition comprising ranpirnase, immunoglobulins, fragments thereof, antibodies, or combinations thereof, obtained from a plasma of a subject immune to said viral disease, and natural killer cells.

A skilled artisan would appreciate that Ranpirnase, called herein also “onconase”, “P-30”, “TMR004”, and “Pannon”, is a ribonuclease enzyme found in the oocytes of the Northern Leopard Frog (Rana pipiens). Ranpirnase is a member of the pancreatic ribonuclease (RNase A) protein superfamily and degrades RNA substrates with a sequence preference for uracil and guanine nucleotides. Ranpirnase has been studied as a potential cancer and antiviral treatment due to its unusual mechanism of cytotoxicity tested against transformed cells and antiviral activity. Ranpirnase UniProt identification number is P85073.

In some embodiments, ranpirnase comprises an amino acid sequence comprising EDWLTFQKKHITNTRDVDCDNIMSTNLFHCKDKNTFIYSRPEPVKAICKGIIASKN VLTTSEFYLSDCNVTSRPCKYKLKKSTNKFCVTCENQAPVHFVGVGSC (SEQ ID No.: 1). In some embodiments, ranpirnase comprises an amino acid sequence comprising at least 80%, 85%, 90%, 95%, or 99% homology to SEQ ID No.:1.

In some embodiments, ranpirnase comprises an amino acid sequence comprising EDWLTFQKKHVTNTRDVDCNNIMSTNLFHCKDKNTFIYSRPEPVKAICKGIIASK NVLTTSEFYLSDCNVTSRPCKYKLKKSTNKFCVTCENQAPVHFVGVGRC (SEQ ID No.: 2). In some embodiments, ranpirnase comprises an amino acid sequence comprising at least 80%, 85%, 90%, 95%, or 99% homology to SEQ ID No.:2.

In some embodiments, ranpirnase comprises an amino acid sequence comprising EDWLTFQKKHITNTRDVDCDNIMSSNLFHCKDKNTFIYSRPEPVKAICKGIIASKN VLTTSEFYLSDCNVTSRPCKYKLKKSTNKFCVTCENQAPVHFVGVGSC (SEQ ID No.: 5). In some embodiments, ranpirnase comprises an amino acid sequence comprising at least 80%, 85%, 90%, 95%, or 99% homology to SEQ ID No.:5.

In some embodiments, a ribonuclease comprises amphinase. In some embodiments, disclosed herein is a composition comprising amphinase and immunoglobulins, fragments thereof, antibodies, or combinations thereof, obtained from a plasma of a subject immune to a viral disease. In some embodiments, disclosed herein is a composition comprising amphinase and immune cells. In some embodiments, disclosed herein is a composition comprising amphinase, immunoglobulins, fragments thereof, antibodies, or combinations thereof, obtained from a plasma of a subject immune to said viral disease, and immune cells. In some embodiments, disclosed herein is a composition comprising amphinase, immunoglobulins, fragments thereof, antibodies, or combinations thereof, obtained from a plasma of a subject immune to said viral disease, and natural killer cells.

A skilled artisan would appreciate that “amphinase”, termed herein also “amphinase 2” and “ramphinase”, is a ribonuclease enzyme found in the oocytes of the Northern leopard frog (Rana pipiens). Amphinase is a member of the pancreatic ribonuclease protein superfamily and degrades long RNA substrates, and has been studied as a potential cancer therapy due to its unusual mechanism of cytotoxicity tested against tumor cells.

In some embodiments, amphinase comprises an amino acid sequence comprising KPKEDREWEKFKTKHITSQSVADFNCNRTMNDPAYTPDGQCKPVNTFIHSTTGP VKEICRRATGRVNKSSTQQFTLTTCKNPIRCKYSQSNTTNFICITCRDNYPVHFVK TGKC (SEQ ID No.: 3). In some embodiments, amphinase comprises an amino acid sequence comprising at least 80%, 85%, 90%, 95%, or 99% homology to SEQ ID No.:3.

In some embodiments, amphinase comprises an amino acid sequence comprising KPKEDREWEKFKTKHITSQSVADFNCNRTMNDPAYTPDGQCKPINTFIHSTTGPV KEICRRATGRVNKSSTQQFTLTTCKNPIRCKYSQSNTTNFICITCRDNYPVHFVKT GKC (SEQ ID No.: 4). In some embodiments, amphinase comprises an amino acid sequence comprising at least 80%, 85%, 90%, 95%, or 99% homology to SEQ ID No.:4.

A skilled artisan would appreciate that ranpirnase and amphinase are RNAse a enzymes. RNAse III enzymes are in the RNAse C family that recognizes double stranded RNA, which the severe acute respiratory syndrome coronavirus 2 (SARS-CoV-2) is not.

A skilled artisan would appreciate that different ribonucleases exert their antiviral activity by different mechanisms. All are relevant to the compositions and methods of the present disclosure. In some embodiments, a ribonuclease enters into the cells via receptor-mediated endocytosis and once internalized into the cytosol, selectively degrades tRNA, resulting in inhibition of protein synthesis and induction of cell apoptosis.

In some embodiments, conjugation, co-encapsulation, or co-formulation of the immunoglobulins disclosed herein and a ribonuclease, results in an anti-viral immunotoxin composition. A skilled artisan will appreciate that ribonucleases are hydrophilic compounds with relatively high stability. In some embodiments, the composition is encapsulated in naturally occurring lipid membranes. In some embodiments, composition is encapsulated in nanoparticles membrane mimetics, as bioxomes.

### Bioxomes

In some embodiments, the compositions disclosed herein are loaded into artificial exosomes. In some embodiments, the ribonucleases disclosed herein are loaded into artificial exosomes. In some embodiments, the immune cells disclosed herein are loaded into artificial exosomes. In some embodiments, the immune cells disclosed herein are co-administered with artificial exosomes.

In some embodiments, the compositions disclosed herein are loaded into a naturally occurring exosomes. In some embodiments, the ribonucleases disclosed herein are loaded into a naturally occurring exosomes. In some embodiments, the immune cells disclosed herein are loaded into a naturally occurring exosomes. In some embodiments, the immune cells disclosed herein are co-administered with a naturally occurring exosomes.

In some embodiments, an artificial exosome, which is termed herein also “bioxosome” having all the same qualities, comprises a cell membrane that undergoes fusion with a target cell and releases its cargo into that target cell after the fusion. In some embodiments, the cell membrane component is derived from a selected cellular or extracellular source. As used herein, the term “bioxome” refers, without limitation to an artificial, submicron nano-particle having resemblance to natural extracellular vesicles (EV).

In some embodiments, the particle size of the bioxome ranges from 0.03 μm to 5 μm. In one embodiment, the size of the bioxome is 0.1-0.7 μm; 0.1-0.5 μm, 0.2-0.5 μm; 0.3-0.5 μm. In another embodiment, the average particle size is 5 μm or less; 1.5 μm or less; 0.7 μm or less; 0.5 μm or less; 0.3 μm or less; 0.15 μm or less. In one embodiment, the average particle size is 0.5 μm to 1.5 μm. In one embodiment, the average particle size is 0.4 μm to 0.8 μm. In another embodiment, the average particle size is 0.3 μm to 0.5 μm. In yet further embodiment, the average particle size is 0.4 μm to 1.5 μm when particle size is measured within few hours after the preparation. In yet another embodiment, the particle size is 0.8 μm to 5 μm when particle size is measured within a month after the preparation and bioxome particles are stored at 0° C. to −4° C.

In one embodiment, the bioxome particles are selective targeting bioxomes. In the context of the invention, the term “selective targeting bioxome” refers, without limitation, to bioxome particles designed for specific targeting ligand or homing moieties. In the selective targeting bioxome of the invention, the ligand or homing moieties are, without limitation, glycosaminoglycan; monospecific or bispecific antibodies; aptamers; receptors; fusion proteins; fusion peptides; or synthetic mimetics thereof; cancer targeting-folic acid; specific phospholipids; cytokines, growth factors; or a combination thereof.

In one embodiment, the membrane of bioxome particles of the invention comprises at least 50% from cell membrane obtained from the cellular source cultured in pre-defined cell culture conditions. In one embodiment, the bioxome particles derived from different sources may show differences in lipid composition compared to the plasma membrane.

In some embodiments, a bioxome comprises extracellular vesicles. In some embodiments, a bioxome comprises extracellular vesicles mimetics. In some embodiments, a bioxome comprises GLM. In some embodiments, a bioxome is formulated into dry submicron powder. In some embodiments, a bioxome is formulated into dry submicron powder prepared by top-down methods, such as milling, extrusion, or grinding, or by bottom up methods, such as liposomes, Bioxomes, spray dry, or freeze-dry.

In some embodiments, the cell membrane component of a bioxome is derived from a selected cellular or extracellular source by a process comprising:
a. Performing total cell lipid extraction from the selected cellular or extracellular source in a mild solvent system to obtain a lipid extract;
b. Drying the lipid extract; and
c. Inducing self-assembly of bioxome particles by performing at least one step of ultrasonication; wherein the resulting bioxome particles in the sample are characterized by an average particle size of 0.03 μm to 5 μm.
   As used herein, the term “mild solvent” refers, without limitation, to any of solvents of Class 3 or of Class 2 with PDE >2.5 mg/day and Concentration limit>250 ppm as defined by the FDA.

In one embodiment, the average particle size is 0.05 μm to 3 μm. In yet another embodiment, the average particle size is 0.08 μm to 1.5 μm. In further embodiment the average particle size is 0.1-0.7 μm; 0.1-0.5 μm, 0.2-0.5 μm; 0.3-0.5 μm. In another embodiment, the average particle size is 5 μm or less; 1.5 μm or less; 0.7 μm or less; 0.5 μm or less; 0.3 μm or less; 0.15 μm or less. In one embodiment, the average particle size is 0.5 μm to 1.5 μm. In one embodiment, the average particle size is 0.4 μm to 0.8 μm. In another embodiment, the average particle size is 0.3 μm to 0.5 μm. In one embodiment, the sample comprising the bioxome particle has the pH of 4.5 to 5. In yet another embodiment, the sample comprising the bioxome particle has the pH of 4.5 to 5. In one embodiment, the solvent system comprises a mixture of polar and non-polar solvents. In one embodiment, the polar solvent in the solvent system is selected from the group consisting of isopropanol, ethanol, n-butanol, and water-saturated n-butanol. In one embodiment, the non-polar solvent in the solvent system is selected from hexane and solvents from the terpene group. In one embodiment, the non-polar solvent in the solvent system is n-hexane. In one embodiment, hexane may be fully or partially suspended by supercritical fluid extraction using supercritical carbon dioxide (scCO2) as a mild “green” solvent has many advantageous properties, including gas-like viscosity, liquid-like density, about 100-fold faster diffusivity than in organic solvents at ambient conditions, as well as operation at relatively low temperature. Terpene/flavonoid may be selected further from alpha-pinene, d-limonene, linalool, eucalyptol, terpineol-4-ol, p-cymene, borneol, delta-3-carene, beta-sitosterol, beta-myrcene, beta-caryophyllene, cannflavin A, apigenin, quercetin and pulegone. In one embodiment, the solvent from the terpene group is selected from the group consisting of d-limonene, a-pinene and para-cymene.

In one embodiment, the polar solvent in the solvent system is isopropanol, and the non-polar solvent is n-hexane. In yet another embodiment, the solvent is Hexane-I sopropanol 3:2 low toxicity solvent mixture. In one embodiment, the solvent system further comprises a stabilizer. In another embodiment, the stabilizer is butyl-hydroxytoluene (BHT). In one embodiment, the solvent system may further comprise additives such as, without limitation, antioxidants, surfactants stabilizers vitamin E, squalene, and cholesterol, or a combination thereof.

In one embodiment, the bioxome engineering is achieved using cell membrane collected from cellular or extracellular source through hydrophilic-hydrophobic self-assembly during cavitation ultrasonication procedure in hydrophilic vehicle. In one embodiment, bioxome particles are extruded after lipid membrane isolation post ultrasonication. In one embodiment, the cargo comprising the active molecules is hydrophilic, and is entrapped into hydrophilic vehicle during ultrasonication, or during extrusion. In yet another embodiment, the cargo is hydrophobic cargo and is entrapped prior to extraction with the solvent system, during extraction, during drying/solvent evaporation procedure, during ultrasonication, during extrusion. Repetitive freeze thawing may improve rate of encapsulation of hydrophilic cargo post drying and post ultrasonication. The level of encapsulation loading is affected by selection of engineering parameter based on sensitivity, stability and desired loading dose of selected cargo as predesigned at each specific therapeutic or research moiety.

In one embodiment, the active molecule, i.e., the ribonuclease may be interwoven into Bioxome core at predefined concentration without risk for viral gene vectors impurities as safety concerns. In one embodiment, the bioxome particles may be electroporated or microinjected. In one embodiment, RNA or DNA may be incorporated into the bioxome particles through gentle ultrasonication at 4° C. in the presence of any suitable protective buffers to maintain integrity of nucleic material for therapeutic delivery. According to the embodiments of the invention, the manufacturing process is compliant with most known industrial features of LNPs and liposomes.

In one embodiment, the cellular or extracellular source for total lipid extraction is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, postnatal umbilical cord, placenta, amniotic sac, kidney tissue, neurological tissue, biological fluids, and excrement or surgery extracted tissues, (i.e. milk, saliva, mucus, blood plasma, urine, feces, amniotic fluids, sebum, postnatal umbilical cord, placenta, amniotic sac, kidney tissue, neurological tissue, adrenal gland tissue, mucosal epithelium, smooth muscle tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue. In yet another embodiment, the lipid extraction is performed from cell-conditioned media, lyophilized conditioned cell media, cell pellet, frozen cells, dry cells, washed cell bulk, non-adhesive cell suspension, and adhesive cell layer.

In yet another embodiment, the cell layer is grown in cell culture plastic ware coated or uncoated by extracellular matrix or synthetic matrix, selected from a (multi) flask, a dish, a scaffold, beads, and a bioreactor. According to one embodiment of the invention, the membrane extract is dried by freeze or/and spray/freeze drying. In yet another embodiment, the membrane extract is dried by evaporation. The evaporation can be carried out by any suitable technique, including, but not limited to speed-vac centrifuge, argon/nitrogen blowdown, spiral air flow and other available solvent evaporation methods in controlled temperature environment, such as microwave or rotor evaporation, Soxhlet extraction apparatus, centrifuge evaporators.

In yet further embodiment, the membrane extract is ultra-sonicated by tip ultra-sonicator in a buffer loaded with desirable active molecules. In one embodiment, the average particle size is 0.4 μm to 1.5 μm when particle size is measured within few hours after the preparation. In yet another embodiment, the particle size is 0.8 μm to 5 μm when particle size is measured within a month after the preparation and bioxome particles are stored at 0° C. to −4° C.

In one embodiment, the bioxome particles are derived from membranes of cellular or extracellular source. In one embodiment, the bioxome particles are engineered on-demand from a pre-defined source. In one embodiment, the cell-source is autologous. The term “autologous” refers to a situation when the donor and the recipient are the same. In one embodiment, the cell-source is non-autologous. In one embodiment the donor source is mesoderm cells including, but not limited to fibroblasts, mesenchymal stem cells, pluripotent and differentiated stem cells, cells of the immune system, dendritic cells, ectoderm, keratinocytes, cells of GI and oral cavity, nasal mucosal cells, neuronal and retinal cells, endothelial cells, cardiospheres, cardiomyocytes, pericytes, and blood cells. In one embodiment, the source for the bioxome particles is stromal cells, keratinocytes, melanocytes, parenchymal cells, mesenchymal stem cells (lineage committed or uncommitted progenitor cells), liver reserve cells, neural stem cells, pancreatic stem cells, and/or embryonic stem cells, bone marrow, skin, liver tissue, pancreas, kidney tissue, neurological tissue, adrenal gland, mucosal epithelium, and smooth muscle.

In one embodiment, bioxomes are loaded with ribonucleases during extraction. In yet another embodiment, the loading is performed during drying, prior to extraction or post. In one embodiment, the obtained bioxome particles may undergo extrusion. In some embodiments, bioxomes are extracted by the HIP extraction system. The advantage of the HIP extraction system of the invention is that in contrast to classic chloroform-methanol lipid extraction, enables extract membrane lipids with minimal lipase activity and directly from/on chloroform-soluble components, such as plastics, cell culture sterile surface wells, including but not limited to hollow fiber, beads, nucleopore, and polycarbonate filters.

For example, HIP would permit direct extraction from polycarbonate is stable in these solvents. HIP extraction can be used for consolation of bioxomes from cells or conditioned medium in parallel with coextraction of RNA or proteins from same cell culture or tissue sample. For such process to cell layer or cell pellet or lyophilized conditioned medium or tissue extract HIP can be premixed with approx ¼-⅕th per volume of water buffer or RNA or DNA or protein stabilizing solution (e.g. RNAsave or Trhaloze or RNAse inhibitor containing buffer). The water phase buffer or stabilizing solution extracts coprecipitated nucleic or protein extract wherein said coextracted nucleic or protein phase then may be separated for example by centrifugation or freezing gradient etc. Such RNA or/and DNA or/and protein containing phase may be further during particle formation with hydrophobic phase of bioxome particle and then used as biotherapeutics or for biomarker diagnostic or research reagent use.

In one embodiment, the process of the invention is compatible with GMP and GLP guidance. In one embodiment, according to the process of the invention, the bioxome particles are harvested from cell biomass; cellular pellet; adhesive cellular layer; medium; or a combination thereof. In one embodiment, the bioxome particles are extracted by single low-toxicity step that allow OECD approved-solvent extraction process. In one embodiment, source cells can be modified prior to the extraction by exposure to mild oxidative stress, starvation, radiation or other in vitro modification of cells in culture, in culture to express more lipophilic antioxidants. In one embodiment the lipophilic anti oxidant is rutin, squalene, tocopherol, retinol, folic acid and derivatives thereof. In one embodiment, the lipid solution component is filter-sterilized. In yet further embodiment, the lipid solution component can be stored in nitrogen or argon at a temperature of −20° C. to −80° C.

In further embodiment, the solvent further comprises detergent surfactant. In one embodiment, the detergent is Polaxomer. In one embodiment, the process comprises lyophilizing/evaporating HIP solvent portion to form a bioxome particle-nucleic acid complex; and ultrasonicating in a hydrophilic carrier/buffer, and/or optional extrusion with desired particle size.

In the embodiments of the invention, the QC specifications for particle size characterization of bioxome particles include, without limitation, the following: particle size; penetration capacity to the target tissues/cells; sterility; non-immunogenicity and safety defined by absence of proteins and nucleic acids. Particle size distribution is measured on Malvern Nano Zetasizer and refined by Zetasizer software. The size of the bioxome particles assemblies are manipulated based on the desired application, making use of commonly available down-sizing techniques. The assemblies may be down-sized by extrusion through membranes with preselected mesh dimensions.

In the context of the invention, the QC specifications for bioxome particles lipid characterization include, without limitation, the following: bioxome particles are qualified and quantified by membrane lipid composition and characteristics, such as: (1) de/saturation index of fatty acids-FA, (2) FA chain length characteristics, (GC; HPLC analytical methods) i.e. Long chain LC-polyunstarurated FA PUFA/medium chain-MC/; (3) polarity (IZON assay); (4) lipid composition, i.e. Content percentage ad/or ratio, e.g. PL-phospholipid composition and ration PC-PE/PI-PS or ratio/percentage between various lipid groups of the Bioxome membranes, e.g. PL/NL (neutral lipid)/CL/GL/TG/FFA (HPLC; TLC; LC-MS; MALDI; column chromatography; etc.); (5) total lipid (vanillin assay, etc.); (6) optional functional lipids and lipid derivatives content, e.g. prostaglandins, prostacyclines, leukotriens, tromboxanes (HPLC; MS-MS; ELISA; RIA; etc.), or (7) metabolites such as hydroxy index- (iodine assay); and (8) ROS mediated oxidation.

In the context of the invention, the QC specifications for final composition comprising bioxome particles include, without limitation, the following: viscosity and osmolarity; pH; number of particles per batch; turbidity; stability specification parameters. Methods of particle measurements and characterization that are provided by IZON Ltd., are also applicable for QC in bioxome particles production.

In the context of the invention, the QC specifications for the bioxome production potency include, without limitation an assay for desired bioxomes activity. For example, cell culture assay to test bioxome and redoxome based products functional effect in vitro. The effect may be screened as QC potency assay by scratch assay, cytotoxicity assay, for example chemotherapeutic drug cytotoxicity assay, ROS generating or hydroxyurea aging inducing assay, inflammation IL19 or TGF beta inducing assay.

### Immune Cells

In some embodiments, disclosed herein is a composition for treating or preventing a viral disease in a subject comprising immunoglobulins, fragments thereof, antibodies, or combinations thereof, obtained from a plasma of a subject immune to a viral disease, and immune cells. In some embodiments, disclosed herein is a composition for treating or preventing a viral disease in a subject comprising plasma of a subject immune to a viral disease, and immune cells. In some embodiments, said viral disease comprise Covid-19.

A skilled artisan would appreciate that immune cells, leukocytes, or white blood cells, comprise cells of the immune system that are involved in protecting the body against both infectious disease and foreign invaders. All white blood cells are produced and derived from multipotent cells in the bone marrow known as hematopoietic stem cells.

In some embodiments, an immune cell is selected from the group comprising neutrophils, eosinophils (acidophiles), basophils, lymphocytes, and monocytes. In some embodiments, a neutrophil is selected from the group comprising segmented neutrophils and banded neutrophils.

In some embodiments, an immune cell comprises a B cell. In some embodiments, an immune cell comprises a memory B cell. In some embodiments, an immune cell comprises a regulatory B cell (Breg). In some embodiments, an immune cell comprises a T cell. In some embodiments, an immune cell comprises a Killer T cell, or cytotoxic T cell. In some embodiments, an immune cell comprises a Helper T cell. In some embodiments, an immune cell comprises a Th1 cell. In some embodiments, an immune cell comprises a Th2 cell. In some embodiments, an immune cell comprises a Regulatory T cell (Treg).

In some embodiments, an immune cell comprises a memory T cell. In some embodiments, an immune cell comprises a Natural Killer (NK) cell. In some embodiments, an immune cell comprises a monocyte. In some embodiments, an immune cell comprises a dendritic cell. In some embodiments, an immune cell comprises a macrophage. In some embodiments, an immune cell comprises a Myeloid dendritic cell (mDC). In some embodiments, an immune cell comprises a plasmacytoid dendritic cell (pDC). In some embodiments, the compositions disclosed herein comprise more than one type of immune cell.

In some embodiments, disclosed herein is a composition for treating or preventing Covid-19 in a subject comprising immunoglobulins, fragments thereof, antibodies, or combinations thereof, obtained from a plasma of a subject immune to Covid-19, and NK cells. In some embodiments, disclosed herein is a composition for treating or preventing a Covid-19 in a subject comprising plasma of a subject immune to Covid-19, and NK cells.

A skilled artisan would appreciate that NK cells can be identified and isolated, for example, by cell surface markers comprising CD16 (FcγRIII), CD57, NKp46. Further, several methods are disclosed in the literature that teach how to isolate and growth NK cells. Any of these methods can be used to produce NK cells for the compositions and methods disclosed herein.

In some embodiments, immune cells comprise human immune cells. In some embodiments, immune cells are obtained from a cell line. In some embodiments, immune cells are obtained from a donor. In some embodiments, immune cells are obtained from a subject immune to a viral disease. In some embodiments, immune cells are obtained from a subject immune to Covid-19.

In some embodiments, disclosed herein is a composition for treating or preventing a viral disease, for example Covid-19 in a subject, comprising stem cells obtained from a plasma of a subject immune to said viral disease.

In some embodiments, stem cells are derived from, liver tissue, adipose tissue, bone marrow, skin, placenta, umbilical cord, Wharton's jelly or cord blood. By “umbilical cord blood” or “cord blood” is meant to refer to blood obtained from a neonate or fetus, most preferably a neonate and preferably refers to blood which is obtained from the umbilical cord or the placenta of newborns.

In some embodiments, a stem cell comprises a mesenchymal stem cell (MSC). These cells can be obtained according to any conventional method known in the art. MSC are defined by expression of certain cell surface markers including, but not limited to, CD 105, CD73 and CD90 and ability to differentiate into multiple lineages including osteoblasts, adipocytes and chondroblasts. MSC can be obtained from tissues by conventional isolation techniques such as plastic adherence, separation using monoclonal antibodies such as STRO-1 or through epithelial cells undergoing an epithelial-mesenchymal transition (EMT).

In some embodiments, adipose tissue-derived stem cells encompass undifferentiated adult stem cells isolated from adipose tissue and may also be term “adipose stem cells”, having all the same qualities and meanings. These cells can be obtained according to any conventional method known in the art.

In some embodiments, placental-derived stem cells encompass undifferentiated adult stem cells isolated from placenta and may be referred to herein as “placental stem cells”, having all the same meanings and qualities.

In some embodiments, stem cells comprises an hematopoietic stem cells (HSCs), which are the stem cells that give rise to other blood cells by haematopoiesis. In some embodiments, HSCs comprises Colony-forming unit-granulocyte-erythrocyte-monocyte-megakaryocyte (CFU-GEMM), Colony-forming unit-lymphocyte (CFU-L), Colony-forming unit-erythrocyte (CFU-E), Colony-forming unit-granulocyte-macrophage (CFU-GM), Colony-forming unit-megakaryocyte (CFU-Meg), Colony-forming unit-basophil (CFU-B), Colony-forming unit-eosinophil (CFU-Eos), or a combination thereof.

Several methods for isolating HSCs are disclosed in the literature, and are thus available to a skilled artisan. Hematopoietic stem cells can be identified or isolated by the use of flow cytometry where the combination of several different cell surface markers (particularly CD34) are used to separate the rare Hematopoietic stem cells from the surrounding blood cells. Hematopoietic stem cells lack expression of mature blood cell markers and are thus, called Lin-. Lack of expression of lineage markers is used in combination with detection of several positive cell-surface markers to isolate Hematopoietic stem cells. In addition, Hematopoietic stem cells are characterised by their small size and low staining with vital dyes such as rhodamine 123 (rhodamine lo) or Hoechst 33342 (side population).

### Immunoglobulins

In some embodiments, the compositions disclosed herein further comprise immunoglobulins, fragments thereof, antibodies, or combinations thereof, obtained from a plasma of a subject immune to a viral disease. In some embodiments, the compositions disclosed herein further comprise plasma of a subject immune to a viral disease. In some embodiments, said viral disease is Covid-19, said immunoglobulins bind SARS-CoV-2, or a combination thereof.

Methods available in the art allow identifying subjects who are infected with a virus, for example CoV or SARS-CoV-2, and recover, mount, or will have mounted, an immune response to these viruses and make IgG or IgM antibodies against them. In one embodiment, these individuals are immune to the viral disease, for example Covid 2019. As a result, their plasma is used in another embodiment as a therapeutic agent to prevent said viral disease, respectively, infection in individuals who are not immune, or as treatment in those subjects who are ill with the disease. In one embodiment, the plasma of immune individuals with immunity to a viral disease is processed to manufacture an immunoglobulin preparation which is effective in preventing and/or treating said viral disease or infection, respectively.

In one embodiment, the SARS-CoV-2 immunoglobulins described herein will supply critical SARS-CoV-2 antibodies, fragments thereof or combinations thereof to subjects who are at risk for this infection. In another embodiment said anti SARS-CoV-2 antibodies, fragments thereof or combinations thereof will be administered to patients who are already ill as a result of this infection.

In one embodiment, the compositions and methods of the invention requires the collection of plasma from subjects who have been exposed to the virus, for example SARS-CoV-2, fragments thereof, its antigen(s), or combinations thereof and the use of said plasma as a therapeutic agent, or further processing of said plasma into therapeutic materials such as immunoglobulins or hyperimmune immunoglobulin preparations, in another embodiment. In one embodiment, the immunoglobulins used in the methods and compositions of the invention, are antibodies, IgG, IgM or a combination thereof.

In one embodiment, the term “antibody” includes complete antibodies (e.g., bivalent IgG, pentavalent IgM), or fragments of antibodies which contain an antigen binding site. Such fragments include in one embodiment Fab, F(ab′)2, Fv and single chain Fv (scFv) fragments. In one embodiment, such fragments may or may not include antibody constant domains. In another embodiment, Fab's lack constant domains which are required for complement fixation. ScFvs are composed of an antibody variable light chain (VL) linked to a variable heavy chain (VH) by a flexible hinge. ScFvs are able to bind antigens and can be rapidly produced in bacteria.

In some embodiments, the current disclosure further includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (VH) and light chain variable domains (VL) which together comprise Fv or scFv, with the addition, in another embodiment, of a heavy chain constant domain (CH1) and a light chain constant domain (CL). The four domains (i.e., VH-CH1 and VL-CL) comprise a Fab. Complete antibodies are obtained in one embodiment, from such a library by replacing missing constant domains once a desired VH-VL combination has been identified.

In one embodiment, the antibody, a fragment thereof, or combinations thereof have sufficiently high affinity and avidity to their target, which may be a viral protein, a peptide, a nucleic acid, a sugar or a combination thereof. In one embodiment the target may be CoV or SARS-CoV-2, or fragments of CoV or SARS-CoV-2, or a combination thereof.

In another embodiment, fractionating the plasma sample, the sample with the immunoglobulins fragments thereof, anti-virus antibodies, or combinations thereof, comprises amplifying the target gene encoding for immunoglobulins fragments thereof, anti-virus antibodies, or combinations thereof. In one embodiment, the terms “amplification” or “to amplify” refer to one or more methods known in the art for copying a target nucleic acid, thereby increasing the number of copies of a selected nucleic acid sequence. Amplification may be exponential in one embodiment, or linear in another. In one embodiment, a target nucleic acid may be either DNA or RNA. The sequences amplified in this manner form an “amplicon.” While the exemplary embodiments described herein relate to amplification using the polymerase chain reaction (“PCR”), numerous other methods are known in the art for amplification of nucleic acids (e.g., isothermal methods, rolling circle methods, etc.) and are considered within the scope of the present invention. The skilled artisan will understand that these other methods may be used either in place of, or together with, PCR methods.

In another embodiment, real time PCR is used in the methods of the invention. The term “real time PCR” refers in one embodiment to the process where a signal emitted from the PCR assay is monitored during the reaction as an indicator of amplicon production during each PCR amplification cycle (i.e., in “real time”), as opposed to conventional PCR methods, in which an assay signal is detected at the endpoint of the PCR reaction. Real time PCR is based in one embodiment on the detection and quantitation of a virus reporter, for example a SARS-CoV-2 reporter. The signal increases in direct proportion to the amount of PCR product in a reaction. By recording the amount of the virus reporter at each cycle, it is possible to monitor the PCR reaction during exponential phase where the first significant increase in the amount of PCR product correlates to the initial amount of target template.

The prevalence of antibodies to a virus, for example SARS-CoV-2, varies considerably among different populations. Plasma will be collected in one embodiment from healthy subjects or pool of subjects who have been previously exposed to the virus, for example SARS-CoV-2, either naturally in one embodiment, or by deliberate vaccination (immunization) in another embodiment, and who have antibodies to the virus in their plasma. These subjects are ascertained in one embodiment from populations where viral infection is high, who have a history of viral infections in the past, who are found to have antibodies to the virus thorough an antibody screening program, who have antibodies as the result of deliberate immunization with the virus or with antigens associated with the virus, or a combination thereof.

In some embodiments, the viral disease comprises Covid-19, and the plasma is collected from:
a. a healthy subject or pool of subjects who have been previously exposed to SARS-CoV-2, naturally or by deliberate immunization, and who have IgG or IgM antibodies to SARS-CoV-2 virus in their plasma; or
b. a subject or pool of subjects where SARS-CoV-2 infection rate is high; or
c. a subject or pool of subjects who have a history of SARS-CoV-2 infection in the past; or
d. a subject or pool of subjects who have antibodies as the result of deliberate immunization with SARS-CoV-2 or with antigens associated with SARS-CoV-2; or
e. any combination thereof.

The processing of subjects (“donors”) shall conform to the regulatory requirements that are applicable in the jurisdiction(s) in which the collections take place. This includes soliciting a medical history and measuring pre-donation parameters (such as blood pressure, temperature, hemoglobin, etc.). In another embodiment, after each donation the collected plasma is screened for markers for transmissible disease (e.g. anti-HIV, anti-HCV, HBsAg, Syphilis, etc.) that are applicable in the jurisdiction(s) in which the collections take place, to minimize the hazard of disease transmission. In one embodiment, all donors are screened for the presence of antibodies to the virus, for example SARS-CoV-2, and in another embodiment, the quantity of antibodies is ascertained.

In one embodiment, the plasma used in the methods and compositions of the invention will be collected from a subject by either plasmapheresis (as source plasma) or after separation from whole blood donations (as recovered plasma). In one embodiment, “plasmapheresis” refers to a process in which the part of the blood, is removed from blood cells by a cell separator. The separator works by either spinning the blood at high speed to separate the cells from the blood, or by passing the blood through a membrane with a cellular sieve, so that only the plasma can pass through. The cells are returned in one embodiment to the person undergoing treatment, while the plasma, which contains the antibodies, is collected.

In one embodiment, the term “recovered plasma” refers to the plasma that is, or has been, separated from whole blood donations. In another embodiment, “recovered plasma” refers to the process whereby heparinized blood is passed through the first filter of a cascade consisting of several filters into a stream containing the corpuscular components and a plasma stream, subjecting the plasma stream to a purification process, recombining the purified plasma and the stream containing the corpuscular particles and reinfusing the recombined blood into the subject. In one embodiment, the purified plasma is recovered, and IgG, IgM, antibodies, their fragments or antigens are removed prior to the recombination of the plasma and the stream containing the corpuscular particles.

After collection, the plasma is frozen in one embodiment, or stored in the liquid state for an appropriate period of time in another embodiment. Conditions of storage will be determined on the basis of optimal preservation of the anti-viral antibodies as well as preventing contamination of the plasma. In one embodiment, usual (frozen) storage and shipping conditions that are applicable to other plasma products are employed for the antibody plasma preparation.

In one embodiment, a concentrated hyperimmune globulin appropriate for use in the treatment or prevention of a viral disease will be prepared from the collected plasma. In another embodiment, the plasma will be pooled in appropriately-sized batches and subjected to a plasma fractionation procedure which will isolate in one embodiment, and/or purify the immunoglobulin fraction and/or anti-viral antibodies from the plasma in other embodiments. This is done in one embodiment by the classical Cohn alcohol precipitation method, or a variant thereof, an ion exchange chromatographic method, an affinity chromatographic method, or any other suitable method such as MS-MS (tandem mass spectrometry), LC-MS (preparatory liquid chromatography and mass spectrometry), crystallization or immunoprecipitation methods etc. in other embodiments. The final material will be concentrated and the titer or quantity of anti-viral antibody adjusted as appropriate. The final material will be sterile and will meet regulatory requirements as applicable in the jurisdiction of manufacture and/or use.

According to this aspect of the invention and in one embodiment, provided is a method of producing a pharmaceutical preparation for the prevention or treatment of a viral disease, for example Covid-2019, comprising: obtaining plasma from a subject immune to the viral disease; pooling said plasma; fractionating said plasma wherein said fractionation isolates or purifies an immunoglobulin, a fragments thereof, an anti-viral antibody, or a combination thereof from the plasma; and concentrating said immunoglobulin, fragments thereof, antibody, or combinations thereof.

In one embodiment, the final material may have a protein concentration of 0.5%-15%. In one embodiment, the protein concentration is between 0.1 and about 1% (w/w) or between about 1 and about 5% (w/w) in another embodiment, or between about 5 and about 10% (w/w) in another embodiment, or between about 10 and about 15% (w/w) in another embodiment. The final formulation may be appropriate for either intravenous, intrapulmonary, intracavitary or intramuscular administration, or both. Shelf life of the materials is ascertained in one embodiment, through appropriate stability studies.

The efficacy of all immunization programs for the prevention and treatment of bacterial or viral infections is based in one embodiment, on the magnitude of circulating antibody levels. Dosing schedules and product specifications are constructed in certain embodiments around the level of antibodies that is generated (in the case of active immunization in one embodiment) or administered (in the case of passive immunization in other embodiments). In one embodiment, Intravenous Immune Globulins (IVIG) are used in patients with primary immune deficiency. These patients are born with hypo- or agammaglobulinemia and are at great risk for life-threatening infection. The life-long monthly administration of IVIG, however, affords these patients a high level of protection against bacterial and viral infections and permits them to live a normal life by providing them, passively, with a broad array of antibody specificities present in a large number of plasmapheresis donors from which the IVIG was manufactured.

### Angiotensin-Converting Enzyme Inhibitor

Angiotensin-converting enzyme (ACE) is a two-domain dipeptidylcarboxypeptidase, involved in the control of blood pressure by hydrolyzing angiotensin I to angiotensin II. ACE also hydrolyzes other substrates such as the bradykinin and the anti-inflammatory peptide N-acetyl-SDKP. Due to this characteristics ACE inhibitors are being used as treatment or prevention of hypertension, heart failure, myocardial infarction and kidney disease.

Drugs approved for cardiovascular diseases, such as beta-blockers, include angiotensin-converting enzyme inhibitors (ACE-I), angiotensin II receptor blockers (ARBs), statins, aspirin, cardiac glycosides and low-molecular-weight heparins (LMWHs).

Angiotensin Converting Enzyme inhibitors (ACE-I) are widely used as a treatment of hypertension and include compounds as: benazepril (Lotensin, Lotensin Hct), captopril (Capoten), enalapril (Vasotec), fosinopril (Monopril), lisinopril (Prinivil, Zestril), moexipril (Univasc), perindopril (Aceon), quinapril (Accupril), ramipril (Altace), and trandolapril (Mavik).

In some embodiments, the compositions disclosed herein further comprise angiotensin-converting enzyme inhibitors (ACE-I). In some embodiments, the composition for treating or preventing a viral disease in a subject comprises a ribonuclease and an ACE-I.

### Viral Diseases

In some embodiments, a viral disease is caused by SARS-CoV-2. In some embodiments, a viral disease is caused by an adenovirus. In some embodiments, a viral disease is caused by a herpesvirus. In some embodiments, a viral disease is caused by a papillomavirus. In some embodiments, a viral disease is caused by a polyomavirus. In some embodiments, a viral disease is caused by a poxvirus. In some embodiments, a viral disease is caused by an hepadnavirus. In some embodiments, a viral disease is caused by a parvovirus. In some embodiments, a viral disease is caused by an astrovirus.

In some embodiments, a viral disease is caused by a calicivirus. In some embodiments, a viral disease is caused by a picornavirus. In some embodiments, a viral disease is caused by a coronavirus. In some embodiments, a viral disease is caused by a flavivirus. In some embodiments, a viral disease is caused by a togavirus. In some embodiments, a viral disease is caused by a hepevirus. In some embodiments, a viral disease is caused by a retrovirus. In some embodiments, a viral disease is caused by an orthomyxovirus. In some embodiments, a viral disease is caused by an arenavirus. In some embodiments, a viral disease is caused by a bunyavirus.

In some embodiments, a viral disease is caused by a filovirus. In some embodiments, a viral disease is caused by a paramyxovirus. In some embodiments, a viral disease is caused by a rhabdovirus. In some embodiments, a viral disease is caused by a reovirus. In some embodiments, a viral disease is caused by Herpes simplex type 1. In some embodiments, a viral disease is caused by Herpes simplex type 2. In some embodiments, a viral disease is caused by Varicella-zoster virus.

In some embodiments, a viral disease is caused by Epstein-Barr virus. In some embodiments, a viral disease is caused by Human cytomegalovirus. In some embodiments, a viral disease is caused by human herpesvirus type 8. In some embodiments, a viral disease is caused by human papillomavirus. In some embodiments, a viral disease is caused by BK virus. In some embodiments, a viral disease is caused by JC virus. In some embodiments, a viral disease is caused by smallpox. In some embodiments, a viral disease is caused by Hepatitis B virus.

In some embodiments, a viral disease is caused by parvovirus B19. In some embodiments, a viral disease is caused by human astrovirus. In some embodiments, a viral disease is caused by Norwalk virus. In some embodiments, a viral disease is caused by coxsackievirus. In some embodiments, a viral disease is caused by hepatitis A virus. In some embodiments, a viral disease is caused by poliovirus. In some embodiments, a viral disease is caused by rhinovirus. In some embodiments, a viral disease is caused by severe acute respiratory syndrome virus. In some embodiments, a viral disease is caused by hepatitis C virus. In some embodiments, a viral disease is caused by yellow fever virus.

In some embodiments, a viral disease is caused by dengue virus. In some embodiments, a viral disease is caused by West Nile virus. In some embodiments, a viral disease is caused by TBE virus. In some embodiments, a viral disease is caused by Rubella virus. In some embodiments, a viral disease is caused by Hepatitis E virus. In some embodiments, a viral disease is caused by Human immunodeficiency virus (HIV). In some embodiments, a viral disease is caused by Influenza virus. In some embodiments, a viral disease is caused by Lassa virus. In some embodiments, a viral disease is caused by Crimean-Congo hemorrhagic fever virus.

In some embodiments, a viral disease is caused by Hantaan virus. In some embodiments, a viral disease is caused by Ebola virus. In some embodiments, a viral disease is caused by Marburg virus. In some embodiments, a viral disease is caused by Measles virus. In some embodiments, a viral disease is caused by Mumps virus. In some embodiments, a viral disease is caused by Parainfluenza virus. In some embodiments, a viral disease is caused by Respiratory syncytial virus. In some embodiments, a viral disease is caused by Rabies virus.

In some embodiments, a viral disease is caused by Hepatitis D. In some embodiments, a viral disease is caused by Rotavirus. In some embodiments, a viral disease is caused by Orbivirus. In some embodiments, a viral disease is caused by Coltivirus. In some embodiments, a viral disease is caused by Banna virus. In some embodiments, a viral disease is caused by more than one virus.

In some embodiments, said viral disease comprises Covid-19. In some embodiments, said viral disease comprises acute hepatitis. In some embodiments, said viral disease comprises AIDS. In some embodiments, said viral disease comprises aseptic meningitis. In some embodiments, said viral disease comprises bronchiolitis. In some embodiments, said viral disease comprises Burkitt's lymphoma. In some embodiments, said viral disease comprises chickenpox. In some embodiments, said viral disease comprises chronic hepatitis.

In some embodiments, said viral disease comprises common cold. In some embodiments, said viral disease comprises congenital rubella. In some embodiments, said viral disease comprises congenital varicella syndrome. In some embodiments, said viral disease comprises congenital seizures in the newborn. In some embodiments, said viral disease comprises croup. In some embodiments, said viral disease comprises cystitis. In some embodiments, said viral disease comprises cytomegalic inclusion disease. In some embodiments, said viral disease comprises fatal encephalitis. In some embodiments, said viral disease comprises gastroenteritis.

In some embodiments, said viral disease comprises German measles. In some embodiments, said viral disease comprises gingivostomatitis. In some embodiments, said viral disease comprises hepatic cirrhosis. In some embodiments, said viral disease comprises hepatocellular carcinoma. In some embodiments, said viral disease comprises herpes labialis. In some embodiments, said viral disease comprises cold sores.

In some embodiments, said viral disease comprises herpes zoster. In some embodiments, said viral disease comprises Hodgkin's lymphoma. In some embodiments, said viral disease comprises hyperplastic epithelial lesions. In some embodiments, said viral disease comprises warts. In some embodiments, said viral disease comprises laryngeal papillomas.

In some embodiments, said viral disease comprises epidermodysplasia verruciformis. In some embodiments, said viral disease comprises infectious mononucleosis. In some embodiments, said viral disease comprises influenza. In some embodiments, said viral disease comprises influenza-like syndrome. In some embodiments, said viral disease comprises Kaposi sarcoma. In some embodiments, said viral disease comprises keratoconjunctivitis.

In some embodiments, said viral disease comprises liver. In some embodiments, said viral disease comprises lung and spleen diseases in the newborn. In some embodiments, said viral disease comprises malignancies. In some embodiments, said viral disease comprises cervical carcinoma. In some embodiments, said viral disease comprises squamous cell carcinomas. In some embodiments, said viral disease comprises measles. In some embodiments, said viral disease comprises multicentric Castleman disease.

In some embodiments, said viral disease comprises mumps. In some embodiments, said viral disease comprises myocarditis. In some embodiments, said viral disease comprises nasopharyngeal carcinoma. In some embodiments, said viral disease comprises pericarditis. In some embodiments, said viral disease comprises pharyngitis. In some embodiments, said viral disease comprises pharyngoconjunctival fever. In some embodiments, said viral disease comprises pleurodynia.

In some embodiments, said viral disease comprises pneumonia. In some embodiments, said viral disease comprises poliomyelitis. In some embodiments, said viral disease comprises postinfectious encephalomyelitis. In some embodiments, said viral disease comprises premature delivery. In some embodiments, said viral disease comprises primary effusion lymphoma. In some embodiments, said viral disease comprises rabies. In some embodiments, said viral disease comprises Reye syndrome.

In some embodiments, said viral disease comprises severe bronchiolitis with pneumonia. In some embodiments, said viral disease comprises skin vesicles. In some embodiments, said viral disease comprises mucosal ulcers. In some embodiments, said viral disease comprises tonsillitis. In some embodiments, said viral disease comprises pharyngitis.

A skilled artisan will recognize that Covid-19, also termed “novel coronavirus pneumonia”, “NCP”, “SARS-CoV-2 acute respiratory disease”, and “COVID-19” comprises an infectious respiratory disease caused by the 2019 novel coronavirus (SARS-CoV-2), which was first detected during the 2019-20 Wuhan coronavirus outbreak. In some embodiments, SARS-CoV-2 is transmitted through human-to-human transmission, generally via respiratory droplets as sneeze, cough or exhalation. In some embodiments, NCP symptoms appear after an incubation period of between 2 to 14 days. In some embodiments, coronavirus primarily affects the lower respiratory tract. In some embodiments, coronavirus primarily affects the upper respiratory tract. In some embodiments, NCP symptoms comprise fever, coughing, shortness of breath, pain in the muscles, tiredness, pneumonia, acute respiratory distress syndrome, sepsis, septic shock, death, or any combination thereof.

A skilled artisan will recognize that SARS-CoV-2 belongs to the broad family of viruses known as coronaviruses. SARS-CoV-2 is a positive-sense single-stranded RNA (+ssRNA) virus. SARS-CoV-2 is a member of the subgenus Sarbecovirus (Beta-CoV lineage B), having an RNA sequence of approximately 30,000 bases in length.

Eighty-one genomes of SARS-CoV-2 had been isolated and reported. The present disclosure comprises compositions and methods for treating these SARS-CoV-2 variants, or any further one.

A skilled artisan will recognize that seven coronavirus types are known to affect humans. The compositions and methods disclosed herein are useful for treating any of them. In some embodiments, coronavirus comprises Human coronavirus 229E (HCoV-229E). In some embodiments, coronavirus comprises Human coronavirus OC43 (HCoV-OC43). In some embodiments, coronavirus comprises Severe acute respiratory syndrome-related coronavirus (SARS-CoV). In some embodiments, coronavirus comprises Human coronavirus NL63 (HCoV-NL63, New Haven coronavirus). In some embodiments, coronavirus comprises Human coronavirus HKU1. In some embodiments, coronavirus comprises Middle East respiratory syndrome-related coronavirus (MERS-CoV), previously known as novel coronavirus 2012 and HCoV-EMC. In some embodiments, coronavirus comprises Novel coronavirus (SARS-CoV-2), also known as Wuhan coronavirus.

In some embodiments, diseases related to CoV comprise common cold, pneumonia, viral pneumonia or a secondary bacterial pneumonia, bronchitis, direct viral bronchitis or a secondary bacterial bronchitis, severe acute respiratory syndrome (SARS), and Middle East respiratory syndrome (MERS).

In some embodiments, the compositions disclosed herein are used to treat or prevent SARS. In some embodiments, the compositions disclosed herein are used to treat or prevent MERS. In some embodiments, the compositions disclosed herein are used to treat or prevent HPV. In some embodiments, the compositions disclosed herein are used to treat or prevent HIV. In some embodiments, the compositions disclosed herein are used to treat or prevent Ebola.

### Autoimmune Disease

Autoimmune disease can develop as a result of a breakdown in immunological tolerance, leading to the activation of self-reactive T cells. There is an established link between infection and human autoimmune diseases. Exogenous pathogen-associated molecular patterns (PAMPs) and endogenous danger signals from necrotic cells bind to pattern recognition receptors (including Toll-like receptors) and activate signalling pathways in innate immune cells and in T cells. This leads to pro-inflammatory cytokine production and T cell activation, which are major factors in the development of autoimmunity. (Mills, 2011)

MicroRNAs (miRNAs) are small conserved non-coding RNA molecules that regulate gene I expression by targeting the 3′ untranslated region (UTR) of specific messenger RNAs (mRNAs). This mechanism is critical for cell cycle, differentiation, and apoptosis. miRNAs also play an important role in the regulation of immunological functions and the prevention of autoimmunity by regulating innate and adaptive immune responses, immune cell development, T regulatory cell stability and function. Differential miRNA expression has been found in rheumatoid arthritis and systemic lupus erythematosus (SLE). (Pauley, Cha, & Chan, 2009) Part of this activity can be attributed to downregulation of NFKB. (Goparaju et al., 2011) It is well known that alterations in the activation of the immune system as the cause of autoimmunity. MicroRNAs have recently been postulated as key posttranscriptional regulators, controlling diverse biological processes, including responses to non-self antigens (Boldin et al., 2011).

### Systemic Lupus Erythematosus (SLE/Lupus)

miRNA biogenesis is a tightly controlled process that requires multiple regulatory mechanisms. miRNAs play a crucial role in maintaining immune system development and function. In SLE, miRNA expression show their clinical importance in SLE pathogenesis. (Liang & Shen, 2012) (Lofgren et al., 2012)

### Rheumatoid Arthritis

Rheumatoid arthritis serves an example of a chronic inflammatory disorder in which miRNAs modulate the inflammatory process in the joints, with the potential to serve as biomarkers for both the inflammatory process and the potential for therapeutic response. (Furer, Greenberg, Attur, Abramson, & Pillinger, 2010).

Alterations in the expression of microRNAs (miRNAs) in regulatory T cells (Tregs) lead to development of autoimmunity in experimental models of autoimmune diseases, such asrheumatoid arthritis (RA). Smigielska-Czepiel and coworkers, used a microarray approach to comprehensively analyze the expression of miRNA in naive Tregs, memory Tregs, and conventional naive and memory T cells (Tconvs) from peripheral blood of RA patients and healthy controls. The authors found a positive correlation between increased expression of miR-451 in T cells of RA patients and disease activity, erythrocyte sedimentation rate levels and serum levels of interleukin-6. The analysis defined miRNAs characteristic for a general naive phenotype and a general memory phenotype that are specifically expressed in both naive and memory Tregs, defining as such miRNA signature characterizing the Treg. (Smigielska-Czepiel et al., 2014).

### Malaria

Artemisinin (Art) and its derivatives are novel antimalarial drugs that exhibit antitumor and antivirus activities. The antitumor effects of combinations of ranpirnase and an Art derivative, dihydroartemisinin (DHA), both in vitro and in vivo Isohologram analyses showed synergistic effects on the proliferation of NSCLC cells under the treatment with ranpirnase and DHA. In vivo experiments also showed that the antitumor effect of ranpirnase was markedly enhanced by DHA in mouse xenograft models. No obvious adverse effect was observed after the treatment. The density of microvasculature in the tumor tissues treated with ranpirnase/DHA combination was lower than those treated with ranpirnase or DHA alone. The above results are consistent with the results of the matrigel plug test for angiogenesis suppression using the ranpirnase/DHA combination. These results imply that the anti-angiogenesis effects may make important contributions to the in vivo antitumor effects of the ranpirnase/DHA combination treatment. (Shen, Li, Ye, Wang, & Fei, 2016).

Without wishing to be bound by any theory, it is hypothesized that the synergistic effect of ranpirnase and DHA can be used for treating malaria. Treatment of malaria using a combination of ranpirnase and antimalarial drugs such as quinine, chloroquine, hydroxychloroquine, and analogues or derivatives thereof, is also contemplated.

### Ranpirnase as an Anti-Inflammatory Agent

Cytokines are regulators of host responses to infection, immune responses, inflammation, and trauma. Some cytokines act to make disease worse (pro-inflammatory), whereas others serve to reduce inflammation and promote healing (anti-inflammatory). Pro inflammatory cytokines can have a deleterious effect (e.g. Interleukin (IL)-1 and tumor necrosis factor (TNF) produce fever, inflammation, tissue destruction, and, in some cases, shock and death). Reducing the biological effects of pro-inflammatory cytokines by agents such as TNF-neutralizing antibodies, soluble TNF receptors, and IL-1 receptor antagonist have been extensively tested and are now an important therapeutic option in autoimmune disorders such as rheumatoid arthritis (Dinarello, 2000).

Systemic lupus erythematosus (SLE) is an autoimmune disease that affects virtually all organ systems. In this context cytokine imbalances contribute to immune dysfunction and inflammation. A major role in this mechanism has been attributed to alterations in the regulation of several inflammatory proteins, such as INF-a, IL-6, IFN-γ, IL-17, IL-21, and IL-2. IL-2 in turn can induce a T-cell phenotype characterized by enhanced B-cell help that can enhance secretion of proinflammatory cytokines and reduce induction of suppressive T cells and activation-induced cell death (Ohl & Tenbrock, 2011).

### Chloroquine Derivatives and Autoimmune Disorders

Antimalarial drugs (e.g. chloroquine and its close structural analogues) were developed primarily to treat malaria; however, they are important therapeutic options in many dermatological, immunological, rheumatological and severe infectious diseases. Chloroquine and hydroxychloroquine have been shown to have various immunomodulatory and immunosuppressive effects in diseases such as lupus erythematosus and rheumatoid arthritis due to their anti-inflammatory and immunomodulatory effects (Al-Bari, 2014).

### Ranpirnase and Inflammation

The expression of inflammatory biomarkers released into the supernatant of cultured mucosal cells treated with ranpirnase was studied. In these experiments, we determined levels of cytokines (GM-CSF, IL-1a, IL-5, IL-7, IL-12/IL-23p40, IL-15, IL-16, IL-17a, TNF-b, VEGF), chemokines (Eotaxin MIP-1B, Eoxtaxin-3, TARC, IP-10, MIP-1a, IL-8-HA, MCP-1, MDC, MCP-4), and pro-inflammatory markers (IFN-y, IL-1, IL-2, IL-4, IL-6, IL-8, IL-10, IL-12p70, IL-13, TNF-a).

These experiments included normal cells and cells infected with HIV.

Of the 30 biomarkers tested, 17 demonstrate a ranpirnase dose-dependent decrease vs infection alone, with dose-dependent defined as two or more consecutive doses demonstrating statistical significance (p<0.05). These include the following members of the chemokine panel: IP-10, MCP-1, MDC, MIP-1 and TARC, and the cytokine panel: IL-1, IL12-p40, IL-7, IL-15 and IL-17, and the pro-inflammatory panel: IFN-, IL12-p70, IL-4, IL-13, IL-1a, IL-8 and IL-5.

The ability of ranpirnase to generate anti-inflammatory responses in colorectal explants was examined in the setting of LPS and demonstrated statistically significant (p<0.05) decreases in 12/30 inflammatory biomarkers tested, including IP-10, MDC, MIP-1a, MIP-1b, TARC, GMCSF, IL-12p40, IL-17, IL-1, TNF, IFN- and IL-12p70 at the highest two doses of 6 and 60 μg/mL.

Synergistic Activity of Ranpirnase Used Together with Chloroquine Derivatives in Treating Various Diseases and Disorders

Chloroquine is a 9-aminoquinoline that has been used since 1934. Besides its antimalarial effects chloroquine has antiviral effects by inhibition of pH-dependent steps of the replication of several viruses like flaviviruses, retroviruses, and coronaviruses. The most studied effects are in HIV replication. Chloroquine has immunomodulatory effects, suppressing tumour necrosis factor a and interleukin 6 (Savarino et al., 2003).

The modulation of inflammatory processes is based on the activity of regulatory T (Treg) cells and suppressive Dendritic Cells (DCs) that prevent the development autoimmune diseases and regulates inflammatory responses. Chloroquine, alters the frequency of Treg cells and DCs in normal mice and also prevents the development of autoimmunity in experimental models of autoimmune encephalomyelitis (an experimental model for human Multiple Sclerosis). (Thom R, et al., 2013) The role of miRNA in Treg cells has also been studied in Multiple sclerosis (MS). T cells (Tregs) play a key role in the autoimmune balance and their improper function may facilitate the expansion of autoaggressive T cell clones. Recently, it has been shown that microRNAs (miRNAs) have been involved in autoimmune disorders and their loss-of-function in immune cells was shown to facilitate systemic autoimmune disorders. The miRNA genome-wide expression profile has been analyzed by microarray on CD4+CD25+high T cells from MS relapsing-remitting patients in stable condition and healthy controls (De Santis et al., 2010).

### Relationship Among Treg Cells and Dicer (a Ranpirnase Ortholog)

Zhou and coworkers studied a new regulatory T (Treg) cell-specific, FoxP3-GFP-hCre bacterial artificial chromosome transgenic mouse that was crossed to a conditional Dicer knockout (KO) mouse strain to analyze the role of microRNAs (miRNAs) in the development and function of Treg cells. Although thymic T reg cells developed normally in this setting, the cells showed evidence of altered differentiation and dysfunction in the periphery. Dicer-deficient Treg lineage cells failed to remain stable, as a subset of cells down-regulated the T reg cell-specific transcription factor FoxP3, whereas the majority expressed altered levels of multiple genes and proteins (including Neuropilin 1, glucocorticoid-induced tumor necrosis factor receptor, and cytotoxic T lymphocyte antigen 4) associated with the Treg cells. In fact, a significant percentage of the Treg lineage cells took on a T helper cell memory phenotype including increased levels of CD127, interleukin 4, and interferon gamma. Importantly, Dicer-deficient Treg cells lost suppression activity in vivo; the mice rapidly developed fatal systemic autoimmune disease resembling the FoxP3 KO phenotype. These results support a central role for miRNAs in maintaining the stability of differentiated Treg cell function in vivo and homeostasis of the adaptive immune system (Zhou et al., 2008).

### Obesity

Obesity-induced chronic inflammation plays a fundamental role in the pathogenesis of metabolic syndrome (MS). Recently, a growing body of evidence supports that miRNAs are largely dysregulated in obesity and that specific miRNAs regulate obesity-associated inflammation. Zhang and coworkers were able to identify an active miRNA-TF-gene regulatory pathways in obesity by detecting differentially expressed genes (DEGs) and differentially expressed miRNAs (DEmiRs) from mRNA and miRNA expression profiles, and by mapping the DEGs and DEmiRs. This group identified an active miRNA-TF-gene regulatory subnetwork in obesity (Zhang, Guo, Chi, Sun, & Chen, 2015).

Potential regulators of adipogenesis include miRNAs, small non-coding RNAs that have been recently shown related to adiposity and differentially expressed in fat tissue. The biologic role of miRNAs expression profile during human adipogenesis was studied by global miRNA expression microarray in human and viral mature miRNAs in human adipocytes during differentiation and in subcutaneous fat samples from non-obese, obese with and without Type-2 Diabetes Mellitus (DM-2) women. Ortega and coworkers detected changes in adipogenesis-related miRNAs and validated them by RT-PCR. This group identified miRNAs significantly deregulated in subcutaneous fat from obese subjects with and without DM-2, and most of these changes were associated with miRNAs also significantly deregulated during adipocyte differentiation (Ortega et al., 2010).

The generation of microRNAs is dependent on the RNase III enzyme Dicer, the levels of which vary in different normal cells and in disease states. It was demonstrated that Dicer protein expression in JAR trophoblast cells, and several other cell types, was inhibited by multiple stresses including reactive oxygen species, phorbol esters and the Ras oncogene. Additionally, double-stranded RNA and Type I interferons repress Dicer protein in contrast to IFN-gamma which induces Dicer. The effects of stresses and interferons are primarily post-transcriptional. The findings suggest that Dicer is a stress response component and identifies interferons as potentially important regulators of Dicer expression (Wiesen & Tomasi, 2009).

miRNAs have a potential role in the central regulation of whole-body energy homeostasis. The expression of Dicer, an essential endoribonuclease for miRNA maturation is modulated by nutrient availability and excess in the hypothalamus. Conditional deletion of Dicer in cells results in obesity, characterized by hyperphagia, increased adiposity, hyperleptinemia, defective glucose metabolism and alterations in the pituitary-adrenal axis. Hypothalamic transcriptomic analysis in Dicer KO mice revealed the downregulation of genes implicated in biological pathways associated with classical neurodegenerative disorders, such as MAPK signaling, ubiquitin-proteosome system, autophagy and ribosome biosynthesis (Schneeberger et al., 2013).

Due to the intense anti-inflammatory activity shown by the expression of anti-inflammatory cytokines and the regulation of miRNA expression induced by ranpirnase.

The present disclosure provides compositions and methods for treating various diseases or disorders including, but not limited to, autoimmune diseases (e.g. rheumatoid arthritis and lupus), malaria, and obesity comprising administering ranpirnase alone or in combination with quinine, chloroquine, hydroxychloroquine, or analogues or derivatives thereof.

In various embodiments, the present disclosure provides methods for treating autoimmune diseases, malaria, and/or obesity in a subject in need thereof, comprising administering to the subject a therapeutically effective dose of ranpirnase.

In embodiments, the present disclosure provides methods for treating autoimmune diseases, malaria, and/or obesity in a subject in need thereof, comprising administering to the subject a therapeutically effective dose of ranpirnase and a therapeutically effective dose of a quinine, chloroquine, hydroxychloroquine, or analogues or derivatives thereof. Ranpirnase and quinine chloroquine, hydroxychloroquine, or analogues or derivatives thereof, may be administered concurrently or before or after administration of each other.

In embodiments, the present disclosure provides methods for modulating fat metabolism in a subject in need thereof, comprising administering to the subject a therapeutically effective dose of ranpirnase, alone or in combination with a therapeutically effective dose of quinine, chloroquine, hydroxychloroquine, or analogues or derivatives thereof. The term “in combination with” as used herein means ranpirnase and quinine, chloroquine, hydroxychloroquine, or analogues or derivatives thereof, may be administered concurrently or before or after administration of each other.

In some embodiments, the compositions disclosed herein further comprise quinine, chloroquine, hydroxychloroquine, or analogues or derivatives thereof. In some embodiments, the composition for treating or preventing a viral disease in a subject comprises a ribonuclease and quinine, chloroquine, hydroxychloroquine, or analogues or derivatives thereof. In some embodiments, the composition for treating or preventing a viral disease in a subject comprises a ribonuclease and quinine. In some embodiments, the composition for treating or preventing a viral disease in a subject comprises a ribonuclease and chloroquine. In some embodiments, the composition for treating or preventing a viral disease in a subject comprises a ribonuclease and hydroxychloroquine. In some embodiments, the composition for treating or preventing a viral disease in a subject comprises a ribonuclease and quinine, chloroquine, hydroxychloroquine.

In embodiments, the present disclosure provides compositions comprising ranpirnase and one or more pharmaceutically acceptable excipients. In some other embodiments, the present disclosure provides compositions comprising ranpirnase and quinine or chloroquine or hydroxychloroquine or analogues or derivatives thereof, and one or more pharmaceutically acceptable excipients. The compositions that contain both active agents, namely, ranpirnase and quinine or chloroquine or hydroxychloroquine or analogues or derivatives thereof, could be in the form of a kit where the kit comprises a first composition comprising ranpirnase and one or more pharmaceutically acceptable excipients and a second composition comprising quinine or chloroquine or hydroxychloroquine or analogues or derivatives thereof, and one or more pharmaceutically acceptableexcipients.

The autoimmune diseases treated by the compositions and methods of the present disclosure include, but are not limited to, rheumatoid arthritis (RA), systemic lupus erythematosus (SLE), juvenile idiopathic arthritis, myasthenia gravis, multiple sclerosis, ankylosing spondylitis, and inflammatory bowel diseases.

### Anti-Viral

The combination of ranpirnase with ranpirnase and quinine or chloroquine or hydroxychloroquine or analogues or derivatives thereof may also be used to treat or prevent viral infection. For example, the virus may be HIV, a coronavirus, ebola, and the like. Exemplary coronaviruses for treating with ribonucleases, such as ranpirnase, alone and m combination with quinine or chloroquine or hydroxychloroquine or analogues or derivatives thereof are MERS, SARS, and COVID-19 (Wuhan). Exemplary Ebola viruses include Marburg strain and Zairestrains.

### Methods of Treatment

Two forms of immunization have been utilized with great success for more than 50 years both for the treatment and prevention of bacterial and viral infections. These are termed active and passive immunization. In some embodiments, the compositions disclosed herein can be used for passive and active immunization.

In one embodiment, active immunization (also called vaccination) involves the administration of either a live, attenuated or killed microorganism, or a portion of said microorganism in order “prime” the cellular immune system and to elicit an antibody response in the subject. Microorganisms may be a bacterium, a virus, a virus-like particle or a combination thereof. The antibody response, which results in certain embodiments, is the ability of the subject's immune system to select, synthesize and secrete antibodies that will kill the specific invading microorganism takes some weeks or months to occur, during which time the subject remains vulnerable to the microorganism. However, once vaccinated, the subject retains the ability to defend himself against that microorganism for part or the rest of his or her life, at least in part by raising specific antibodies against the microorganism when exposed. (although booster immunizations may be required periodically). Active immunization has been shown to be highly effective in conferring long-term protection against certain conditions and is generally administered when the subject is well and has not been recently exposed to the inoculum. Examples of active viral vaccines include smallpox, polio, and hepatitis B.

Passive immunization involves in another embodiment, the administration to the subject of a purified immunoglobulin preparation which contains relatively high quantities of one or more antibodies specific to the target microorganism. In one embodiment, passive administration of such antibodies confers immediate but temporary immunity against a specific microorganism, usually for the time that the antibodies are present in the body (perhaps a month or two). As a result, passive immunization is used when the subject has been recently exposed to a specific microorganism or is at high risk of being exposed to a microorganism in an attempt to prevent, or modify the severity of, disease caused by the microorganism in question. Examples of viral passive antibodies given prophylactically include Rabies immunoglobulin and Varicella-Zoster immunoglobulin. In some cases, passive immunization is given when the subject is already ill, as a therapeutic agent. Examples of passive immunization include but are not limited to viral antibodies given therapeutically, include Hepatitis B immunoglobulin in liver transplants for Hepatitis B liver failure and Cytomegalovirus immunoglobulin. These therapies have proven to be highly effective as well.

In one embodiment, the compositions of the invention are used in the methods of the invention described herein. In one embodiment, the invention provides a method of preventing or treating a viral disease, for example Covid-2019 in a subject, comprising any of the compositions disclosed herein. In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

As used herein, “subject” refers in one embodiment, to a human or any other animal which has been exposed to and is now immune to CoV related disease or Covid-2019. A subject refers to a human presenting to a medical provider for diagnosis or treatment of a disease, such as a CoV related disease or Covid-2019 in another embodiment. A human includes pre- and postnatal forms. In one embodiment, subjects are humans being treated for symptoms associated with a CoV related disease or Covid-2019, or a volunteer for hyperimmune antibody production following the volunteer's exposure to an attenuated virus or the like.

In some embodiments, an extracorporeal device is used to deliver the pharmaceutical composition. In some embodiments, the patient blood is previously cleaned by antiviral phototherapy. In some embodiments, phototherapy comprises antiviral agents such as methylene blue, rose Bengal, carbon dot, quantum dot, activated photosensors or a combination thereof. In some embodiments, plasmapheresis comprises cutting off viral particles with TTF ultrafiltration or by hollow fiber filtration exposed to carbon dot and/or other phototherapy synergistic enhancers.

The term “therapeutically effective amount” or “effective amount” refers in one embodiment, to an amount of a monovalent or combination vaccine sufficient to elicit a protective immune response in the subject to which it is administered. The immune response may comprise, without limitation, induction of cellular and/or humoral immunity.

The amount of a vaccine that is therapeutically effective may vary depending on the particular antibody used in the vaccine, the age and condition of the subject, and/or the degree of infection, and can be determined by an attending physician.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In another embodiment, a controlled release system can be placed in proximity to the therapeutic target, i.e., the brain, thus requiring only a fraction of the systemic dose.

Such compositions are in one embodiment liquids or lyophilized or otherwise dried 30 formulations and include diluents of various buffer content (e.g., Tris-HCl·, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexion with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, virosomes, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral, as well as self-administration devices.

The dosage regimen for treating a condition with the compositions of this invention is selected in one embodiment, in accordance with a variety of factors, such as the type, age, weight, ethnicity, sex and medical condition of the subject, the severity of the condition treated, the route of administration, and the particular compound employed, and thus may vary widely while still be in the scope of the invention.

### Pharmaceutical Compositions

In one embodiment, the pharmaceutical preparation of the invention, used in the methods of the invention comprise a carrier, excipient, flow agent, processing aid, a diluent, or a combination thereof.

In one embodiment, the compositions used in the invention further comprise a carrier, or excipient, lubricant, flow aid, processing aid or diluent in other embodiments, wherein the carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal ophthalmic and oral. In one embodiment the pharmaceutical composition is administered parenterally, transmucosally, transdermally, intramuscularly, intravenously, intradermally, subcutaneously, intraperitonealy, intraventricularly, or intracranially.

In the methods of the present invention, ranpirnase may be administered systemically or topically. Systemic administration includes oral and intravenous routes of administration. Topical administration routes includes application to the skin (cutaneous), inhalational, ophthalmic, otic, and application to the mucosal membrane.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In some embodiments, the compositions disclosed herein are encapsulated within extracellular vesicles (Evs), or mimics thereof, or submicron particles, according to prior art known technologies. In some embodiments, the pharmaceutical composition is formulated as a sterile lyophilizate. In some embodiments, the lyophilizate of immunoglobulins is preferably encapsulated within extracellular vesicles—Evs, or mimics thereof, or submicron particles.

In some embodiments, prior to administration, the lyophilizate is resuspended aseptically in a sterile buffer. In some embodiments, a physiologically tolerated buffer is added to facilitate pH control. In some embodiments, the formulations of the present invention have pH between about 6.8 and about 7.8. In some embodiments, buffers include phosphate buffers, sodium phosphate, or phosphate buffered saline (PBS). In some embodiments, the final product lyophilizate may include preformulated isotonicity agents as glycerin, stabilizers excipients, such as carbohydrates (trehaloze, sucrose), an antioxidant, a chelating agent, such as EDTA and EGTA, human serum albumin, or a combination thereof, which can optionally be added to the formulations or compositions to reduce aggregation. Surfactants additives are particularly useful if a pump or plastic container is used to administer the formulation. An optional carrier additive is human serum albumin, or an enhancer surfactant as described below. The presence of pharmaceutically acceptable surfactant mitigates the propensity of proteins to aggregate. Such acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), block co-polymers known in the state of the art. Further, glycerol monolaurate—GML, an approved pharmaceutical excipient surfactant, can be used at concentrations up to 3 mg/ml (which is similar to the amount of GML in human milk) to prepare enhanced delivery system to penetrate cell barriers if administered orally or intramucosally.

In some embodiments, the composition disclosed herein is administered in combination with plasmapheresis collected from a healthy donor. In some embodiments, the composition disclosed herein is delivered by a special extracorporeal device. In some embodiments, the composition disclosed herein is administered together with platelet-derived extracellular vesicles (EVs) or mimics of thereof. In another embodiment, the composition disclosed herein is synergistically combined with cellular and acellular components obtained from placental tissue and/or placental perfusate. In some embodiments, placental cells combined with the pharmaceutical composition comprise hematopoietic (CD34.sup.+) cells, nucleated cells such as granulocytes, monocytes and macrophages, a small percentage (less than 1%) of substrate-adherent placental stem cells, and natural killer cells.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

In some embodiments, a synergistic effect is attained by delivering the pharmaceutical composition by various routes of administration, such as intravenous, intravesicular, oral, nasal, intraperitoneal, intrapulmonary (inhalation), intramuscular, subcutaneous, intra-tracheal, transmucosal, or transdermal route. In some embodiments, osmotic or micro pumps are used to deliver the pharmaceutical composition by any of these routes. In some embodiments, the compositions are formulated as a gel or spray.

A skilled artisan will appreciate that in some embodiments, an optimal dose for a route of administration is determined by a biosensing complex comprising a mixture of polydopamine (PDA) and protein G. PDA is a representative mussel-inspired polymer, and protein G is an immunoglobulin-binding protein that enables an antibody to have an optimal orientation.

In one embodiment, the pharmaceutical composition disclosed herein further comprises an additional therapeutic agent, a vaccine, an adjuvant or a combination thereof.

Adjuvants suitable for use in the compositions and methods described herein include, but are not limited to several adjuvant classes such as; mineral salts, e.g., Alum, aluminum hydroxide, aluminum phosphate and calcium phosphate; surface-active agents and microparticles, e.g., nonionic block polymer surfactants (e.g., cholesterol), virosomes, saponins (e.g., Quil A, QS-21, Alum and GPI-0100), proteosomes, immune stimulating complexes, cochleates, quaternary amines (dimethyl diocatadecyl ammonium bromide (DDA)), pyridine, vitamin A, vitamin E; bacterial products such as the RIBI adjuvant system (Ribi Inc.), cell wall skeleton of Mycobacterium phlei (Detox®), muramyl dipeptides (MDP) and tripeptides (MTP), monophosphoryl lipid A, Bacillus Calmete-Guerin (BCG), heat labile E. coli enterotoxins, cholera toxin, trehalose dimycolate, CpG oligodeoxnucleotides; cytokines and hormones, e.g., interleukins (IL-1, IL-2, IL-6, IL-12, IL-15, IL-18), granulocyte-macrophage colony stimulating factor, dehydroepiandrosterone, 1,25-dihydroxy vitamin D3; polyanions, e.g., dextran; polyacrylics (e.g., polymethylmethacrylate, Carbopol 934P); carriers e.g., tetanus toxid, diptheria toxoid, cholera toxin B subunit, mutant heat labile enterotoxin of enterotoxigenic E. coli (rmLT), heat shock proteins; oil-in-water emulsions e.g., AMPHIGEN® (Hydronics, USA); and water-in-oil emulsions such as, e.g., Freund's complete and incomplete adjuvants.

In some embodiments, the pharmaceutical compositions disclosed herein can be administered with a further antiviral compound. In some embodiments, said further antiviral compounds enhance the effect of the pharmaceutical compositions synergistically.

In some embodiments, said further antiviral agents are selected from a group comprising an antiviral enzyme or an approved antiviral drug, acyclovir, valaciclovir, famciclovir, sofosbuvir, ribavirin, pegylated interferon-.alpha.-2a, pegylated interferon-.alpha., penciclovir.-2b, boceprevir, telaprevir, ledipasvir, and simiprevir, nucleoside reverse transcriptase inhibitors, non-nucleoside reverse transcriptase inhibitors, protease inhibitors, and fusion inhibitors, valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1'S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-q-uinolinecarboxamine (PNU-183792), 2-bromo-5,6-dichloro-1-(.beta.-D-ribofuranosyl)benzimidazole (BDCRB), maribavir, 3-hydroxy-2,2-dimethyl-N-[4-{[(5-dimethylamino)-1-naphthyl]-sulfonyl]-ami-no)phenyl}propamide (BAY 38-4766), N—[N-[4-(2-aminothiazol-4-yl)phenyl]carbamoylmethyl]-N-[1(S)-phenylethyl-]pyridine-4-carboxamide (BILS179BS), N-[5-(aminosulfonyl)-4-methyl-1,3-thiazol-2-yl]-N-methyl-2-{4-(2-pyridiny-1)phenyl}acetamide (BAY 57-1293), 2H-3-(4-chlorophenyl)-3,4-dihydro-1,4-benzo-thiazine-2-carbonitrile 1,1-dioxide, 2-chloro-3-pyridin-3-yl-5,6,7,8-tetrahydronindolizine-1-carboxamide (CMV423), or any combination thereof.

In some embodiments, a synergistic enhancer is selected from a group comprising hydroxyurea, leflunomide, EGCG, CBD, squalamine or aminosterol, mycophenolic acid, resveratrol, or a combination thereof. In some embodiments, these synergistic enhancers enhance the effects of the pharmaceutical composition in a synergistic manner.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more. rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, in addition to the immunoglobulins fragments thereof, anti-viral antibodies, or combinations thereof, used in the pharmaceutical preparations of the invention, which in another embodiment are used in the methods of the invention, the pharmaceutical preparations comprise a vaccine comprising nucleic acids encoding polypeptides of the respective virus.

### Delivery by Aerosol Spray

In some embodiments, the compositions disclosed herein are delivered by aerosol. In some embodiments, the compositions are formulated considering the particle size distribution of aerosol used to deliver it. A skilled artisan will appreciate that the aerodynamic particle size distribution is influenced by the characteristics of the spray of the drug product and engineering parameters of the delivery device. Further, particles formulation should be designed to avoid aggregation, improve aerodynamic flowability and reach high uniformity of particle size.

Particle size below 5 microns can reach bronchi and lungs, while the particles between 5-10 microns are suitable for nasal delivery. A skilled artisan will appreciate that particles above 10 microns are usually not suitable for inhalation, as they are swallowed and go directly into gastrointestinal tract. In some embodiments, particle size is in a range of about 0.1 micron and 5 micron. In some embodiments, particle size is in a range of about 0.2 micron and 1 micron. In some embodiments, a range of about 0.2 micron and 1 micron allows the particles to reach the lungs. In some embodiments, particle size is in a range of about 0.5 micron and 1.5 micron. In some embodiments, a range of about 0.5 micron and 1.5 micron allows the particles to be absorbed by transmucosal delivery.

Glycerol monolaurate (GML) is a natural surfactant permeability enhancer that can 15 be used for transmucosal delivery of ribonucleases to enhance its anti-viral bioactivity. In some embodiments, GML is used at its critical micelles concentrations to prepare the composition in the presence of carbohydrate/HSA stabilizers an aqueous base. In some embodiments, the size of these nanospheres for transmucosal delivery is smaller than 100 nm.

In some embodiments, a protein compatible solvent comprising an optional gamma irradiation dose (5-20 kGy at the rate of more than 1 kGy per hour) is used for the preparation of the compositions disclosed herein. In some embodiments, using said solvents results in high encapsulation concentration of protein in GML/stabilizer matrix, and the production of small size nanoparticles. A skilled artisan will appreciate that these nanoparticles can enter the alveoli of the lungs by deep intramucosal delivery.

In some embodiments, ranpirnase is administered systemically, alone or in combination with quinine, chloroquine, hydroxychloroquine, or analogues or derivatives thereof, at a dose of about 5 μg/m² to about 960 μg/m², including values and ranges therebetween. For example, ranpirnase is administered systemically at a dose of about 5, 8, 10, 12, 15, 18, 20, 22, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, or 960 μg/m², including values and ranges between.

In some embodiments, ranpirnase is administered systemically, alone or in combination with quinine, chloroquine, hydroxychloroquine, or analogues or derivatives thereof, at a dose of about 5 μg/m² to about 960 μg/m², about 5 μg/m² to about 920 μg/m², about 5 μg/m² to about 900 μg/m², about 5 μg/m² to about 850 μg/m², about 5 μg/m² to about 800 μg/m², about 5 μg/m² to about 750 μg/m², about 5 μg/m² to about 700 μg/m², about 5 μg/m² to about 650 μg/m², about 5 μg/m² to about 600 μg/m², about 5 μg/m² to about 550 μg/m², about 5 μg/m² to about 500 μg/m², about 5 μg/m² to about 450 μg/m², about 5 μg/m² to about 400 μg/m², about 5 μg/m² to about 350 μg/m², about 5 μg/m² to about 300 μg/m², about 5 μg/m² to about 250 μg/m², about 5 μg/m² to about 200 μg/m², about 5 μg/m² to about 150 μg/m², about 5 μg/m² to about 100 μg/m², about 5 μg/m² to about 50 μg/m² including values and ranges therebetween.

In some embodiments, ranpirnase is administered systemically, alone or in combination with quinine, chloroquine, hydroxychloroquine, or analogues or derivatives thereof, at a dose of about 5 μg/m² to about 400 μg/m², 10 μg/m² to about 400 μg/m², 10 μg/m² to about 350 μg/m², 10 μg/m² to about 300 μg/m², 10 μg/m² to about 250 μg/m², 10 μg/m² to about 200 μg/m², 10 μg/m² to about 150 μg/m², 10 μg/m² to about 100 μg/m², 10 μg/m² to about 50 μg/m², 15 μg/m² to about 400 μg/m², 15 μg/m² to about 350 μg/m², 15 μg/m² to about 300 μg/m², 15 μg/m² to about 250 μg/m², 15 μg/m² to about 200 μg/m², 15 μg/m² to about 150 μg/m², 15 μg/m² to about 100 μg/m², 15 μg/m² to about 50 μg/m², 20 μg/m² to about 400 μg/m², 20 μg/m² to about 350 μg/m², 20 μg/m² to about 300 μg/m², 20 μg/m² to about 250 μg/m², 20 μg/m² to about 200 μg/m², 20 μg/m² to about 150 μg/m², 20 μg/m² to about 100 μg/m², 20 μg/m² to about 50 μg/m², 25 μg/m² to about 400 μg/m², 25 μg/m² to about 350 μg/m², 25 μg/m² to about 300 μg/m², 25 μg/m² to about 250 μg/m², 25 μg/m² to about 200 μg/m², 25 μg/m² to about 150 μg/m², 25 μg/m² to about 100 μg/m², 25 μg/m² to about 75 μg/m², 25 μg/m² to about 50 μg/m², 30 μg/m² to about 400 μg/m², 30 μg/m² to about 350 μg/m², 30 μg/m² to about 300 μg/m², 30 μg/m² to about 250 μg/m², 30 μg/m² to about 200 μg/m², 30 μg/m² to about 150 μg/m², 30 μg/m² to about 100 μg/m², 30 μg/m² to about 75 μg/m², 30 μg/m² to about 60 μg/m², 40 μg/m² to about 400 μg/m², 40 μg/m² to about 350 μg/m², 40 μg/m² to about 300 μg/m², 40 μg/m² to about 250 μg/m², 40 μg/m² to about 200 μg/m², 40 μg/m² to about 150 μg/m², 40 μg/m² to about 100 μg/m², 50 μg/m² to about 400 μg/m², 50 μg/m² to about 300 μg/m², 50 μg/m² to about 250 μg/m², 50 μg/m² to about 200 μg/m², 50 μg/m² to about 150 μg/m², 50 μg/m² to about 100 μg/m², 75 μg/m² to about 400 μg/m², 75 μg/m² to about 350 μg/m², 75 μg/m² to about 300 μg/m², 75 μg/m² to about 250 μg/m², 75 μg/m² to about 200 μg/m², 75 μg/m² to about 150 μg/m², 100 μg/m² to about 400 μg/m², 100 μg/m² to about 350 μg/m², 100 μg/m² to about 300 μg/m², 100 μg/m² to about 250 μg/m², 100 μg/m² to about 200 μg/m², 150 μg/m² to about 400 μg/m², 150 μg/m² to about 350 μg/m², 150 μg/m² to about 250 μg/m², or 200 μg/m² to about 300 μg/m², including values and ranges therebetween.

In some embodiments, ranpirnase is administered topically, alone or in combination with quinine, chloroquine, hydroxychloroquine, or analogues or derivatives thereof. In certain embodiments, ranpirnase is administered topically in the form of a topical composition comprising about 0.01% by weight to about 10% by weight of ranpirnase, based on the total weight of the topical composition. In some other embodiments, ranpirnase is administered topically in the form of a topical composition comprising about 0.1% by weight to about 1% by weight of ranpirnase, based on the total weight of the topical composition. In yet some other embodiments, ranpirnase is administered topically in the form of a topical composition comprising about 1% by weight of ranpirnase, based on the total weight of the topical composition.

In embodiments, the concentration of ranpirnase in the topical formulation ranges from about 0.1% to about 10% w/w, such as, about 0.1%, 0.2%, 0.3%, 0.4%, 0.5%, 0.6%, 0.7%, 0.8%, 0.9%, 1.0%, 2%, 3%, 4%, 5%, 6%, 7%, 8%, 9%, or 10% w/w, including values and ranges therebetween.

In some embodiments, the concentration of ranpirnase in the topical formulation ranges from about 0.1% to about 10% w/v, such as, about 0.1%, 0.2%, 0.3%, 0.4%, 0.5%, 0.6%, 0.7%, 0.8%, 0.9%, 1.0%, 2%, 3%, 4%, 5%, 6%, 7%, 8%, 9%, or 10% w/v, including values and ranges therebetween.

In some embodiments, the concentration of ranpirnase in the topical formulation ranges from about 0.1 mg/mL to about 10 mg/mL, such as, about 0.1, 0.2, 0.3, 0.4, 0.5, 0.6, 0.7, 0.8, 0.9, 1.0, 2, 3, 4, 5, 6, 7, 8, 9, or 10 mg/mL, including values and ranges therebetween.

In some embodiments, hydroxychloroquine is dosed as follows. Hydroxychloroquine (HCQ) is available commercially as Plaquenil.

### Exemplary Hydroxychloroquine Dosing Information:

### Adult Dose for Malaria:

Treatment of the acute attack: 800 mg (620 mg base) followed in 6 to 8 hours by 400 mg (310 mg base), then 400 mg (310 mg base) once a day for 2 consecutive days; alternatively, a single dose of 800 mg (620 mg base) has also been effective.

### Dosage on the Basis of Body Weight:

First dose: 10 mg base/kg (not to exceed 620 mg base)
Second dose: 5 mg base/kg (not to exceed 310 mg base) 6 hours after first dose.
Third dose: 5 mg base/kg 18 hours after second dose
Fourth dose: 5 mg base/kg 24 hours after third dose

### Adult Dose for Malaria Prophylaxis:

Suppression: 400 mg (310 mg base) orally on the same day every week

Suppressive therapy should begin 2 weeks prior to exposure; however, failing this, an initial dose of 800 mg (620 mg base) may be taken in 2 divided doses (6 hours apart). Suppressive therapy should continue for 8 weeks after leaving the endemic area.

### Adult Dose for Rheumatoid Arthritis:

Initial dose: 400 to 600 mg (310 to 465 mg base) orally once a day Maintenance dose: 200 to 400 mg (155 to 310 mg base) orally once a day

### Adult Dose for Systemic Lupus Erythematosus:

### Discoid and Systemic Lupus Erythematosus:

Initial dose: 400 mg (310 mg base) orally once or twice a day for several weeks or months, depending on patient response.
Maintenance dose: 200 to 400 mg (155 to 310 mg base) orally once a day

### Pediatric Dose for Malaria:

Treatment of the acute attack: 1 year or older:
First dose: 10 mg base/kg (not to exceed 620 mg base)
Second dose: 5 mg base/kg (not to exceed 310 mg base) 6 hours after first dose
Third dose: 5 mg base/kg 18 hours after second dose
Fourth dose: 5 mg base/kg 24 hours after third dose.

### Pediatric Dose for Malaria Prophylaxis:

### Suppression:

1 year or older: 5 mg base/kg of body weight (not to exceed 310 mg base) orally on the same day every week.

Suppressive therapy should begin 2 weeks prior to exposure; however, failing this, an initial dose of 10 mg base/kg (not to exceed 620 mg base) may be taken in 2 divided doses (6 hours apart).

Suppressive therapy should continue for 8 weeks after leaving the endemic area.

In some embodiments, hydroxychloroquine (base) is administered at a dose of about 100 to about 800 mg, about 100 to about 700 mg, about 100 to about 600 mg, about 100 to about 500 mg, about 100 to about 400 mg, about 100 to about 300 mg, about 200 to about 800 mg, about 200 to about 600 mg, about 200 to about 500 mg, about 200 to about 400 mg, about 300 to about 800 mg, about 300 to about 700 mg, about 300 to about 600 mg, about 300 to about 500 mg, including values and ranges therebetween, daily, every other day, every two days, every three days, weekly, or bi-weekly. In some other embodiments, hydroxychloroquine (base) is administered at a dose of about 100,150,200, 250, 300, 350, 400, 450, 500, 550, 600, 650, 700, 750, 800, 850, or about 900 mg, including values and ranges therebetween, daily, every other day, every two days, every three days, weekly, or bi-weekly.

In some embodiments, chloroquine is dosed as follows.

### Exemplary Chloroquine Dosing Information:

### Adult Dose for Malaria Prophylaxis

500 mg chloroquine phosphate (300 mg base) orally on the same day each week
If possible, suppressive therapy should start 2 weeks prior to exposure; if unable to start 2 weeks before exposure, an initial loading dose of 1 g chloroquine phosphate (600 mg base) may be taken orally in 2 divided doses, 6 hours apart.
Suppressive therapy should continue for 8 weeks after leaving the endemic area.

### CDC Recommendations:

300 mg base (500 mg salt) orally once a week

### Adult Dose for Malaria

60 kg or more: 1 g chloroquine phosphate (600 mg base) orally as an initial dose, followed by 500 mg chloroquine phosphate (300 mg base) orally after 6 to 8 hours, then 500 mg chloroquine phosphate (300 mg base) orally once a day on the next 2 consecutive days
Total dose: 2.5 g chloroquine phosphate (1.5 g base) in 3 days
Less than 60 kg:
First dose: 16.7 mg chloroquine phosphate/kg (10 mg base/kg) orally
Second dose (6 hours after first dose): 8.3 mg chloroquine phosphate/kg (5 mg base/kg) orally
Third dose (24 hours after first dose): 8.3 mg chloroquine phosphate/kg (5 mg base/kg) orally
Fourth dose (36 hours after first dose): 8.3 mg chloroquine phosphate/kg (5 mg base/kg) orally Total dose: 41.7 mg chloroquine phosphate/kg (25 mg base/kg) in 3 days

### Pediatric Dose for Malaria Prophylaxis

Infants and children: 8.3 mg chloroquine phosphate/kg (5 mg base/kg) orally on the same day each week
Pediatric dose should not exceed the adult dose regardless of weight.
If possible, suppressive therapy should start 2 weeks prior to exposure; if unable to start 2 weeks before exposure, an initial loading dose of 16.7 mg chloroquine phosphate/kg (10 mg base/kg) may be taken orally in 2 divided doses, 6 hours apart.
Suppressive therapy should continue for 8 weeks after leaving the endemic area.

### Pediatric Dose for Malaria

### Infants and Children:

Less than 60 kg:
First dose: 16.7 mg chloroquine phosphate/kg (10 mg base/kg) orally
Second dose (6 hours after first dose): 8.3 mg chloroquine phosphate/kg (5 mg base/kg) orally Third dose (24 hours after first dose): 8.3 mg chloroquine phosphate/kg (5 mg base/kg) orally Fourth dose (36 hours after first dose): 8.3 mg chloroquine phosphate/kg (5 mg base/kg) orally Total dose: 41.7 mg chloroquine phosphate/kg (25 mg base/kg) in 3 days
60 kg or more:
First dose: 1 g chloroquine phosphate (600 mg base) orally
Second dose (6 hours after first dose): 500 mg chloroquine phosphate (300 mg base) orally
Third dose (24 hours after first dose): 500 mg chloroquine phosphate (300 mg base) orally
Fourth dose (36 hours after first dose): 500 mg chloroquine phosphate (300 mg base) orally Total dose: 2.5 g chloroquine phosphate (1.5 g base) in 3 days

### CDC Recommendations:

Chloroquine-sensitive uncomplicated malaria (Plasmodium species or species not identified): 10 mg base/kg orally at once, followed by 5 mg base/kg orally at 6, 24, and 48 hours
Total dose: 25 mg base/kg

In some embodiments, chloroquine (base) is administered at a dose of about 100 to about 1200 mg, about 100 to about 1100 mg, about 100 to about 1000 mg, about 100 to about 900 mg, about 100 to about 800 mg, about 100 to about 700 mg, about 100 to about 600 mg, about 100 to about 500 mg, about 100 to about 400 mg, about 200 to about 1200 mg, about 200 to about 1100 mg, about 200 to about 1000 mg, about 200 to about 900 mg, about 200 to about 800 mg, about 200 to about 700 mg, about 300 to about 1200 mg, about 300 to about 1100 mg, about 300 to about 1000 mg, about 300 to about 900 mg, about 300 to about 800 mg, about 300 to about 700 mg, about 400 to about 1200 mg, about 400 to about 1100 mg, about 400 to about 1000 mg, about 400 to about 900 mg, about 400 to about 800 mg, about 500 to about 1200 mg, about 500 to about 1100 mg, about 500 to about 1000 mg, including values and ranges therebetween, daily, every other day, every two days, every three days, weekly, or bi-weekly. In other embodiments, chloroquine (base) is administered at a dose of about 50, 100, 150, 200, 250, 300, 350, 400, 450, 500, 550, 600, 650, 700, 750, 800, 850, 900, 950, 1000, 1050, 1100, 1150, 1200 mg, including values and ranges therebetween, daily, every other day, every two days, every three days, weekly, or bi-weekly.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### REFERENCES

Al-Bari, A. A. (2014). Chloroquine analogues in drug discovery: New directions of uses, mechanisms of actions and toxic manifestations from malaria to multifarious diseases. Journal of Antimicrobial Chemotherapy, 70(6), 1608-1621. http://doi.org/10.1093/jac/dkv018
Ardelt, W., Ardelt, B., & Darzynkiewicz, Z. (2009). Ribonucleases as potential modalities in anticancer therapy. European Journal of Pharmacology, 625(1-3), http://doi.org/10.1016/j.ejphar.2009.06.067 181-189
Boldin, M. P., Taganov, K. D., Rao, D. S., Yang, L., Zhao, J. L., Kalwani, M., Baltimore, D. 2011). miR-146a is a significant brake on autoimmunity, myeloproliferation, and cancer in mice. The Journal of Experimental Medicine, 208(6), 1189-201. http://doi.org/10.1084/jem.20101823
De Santis, G., Ferracin, M., Biondani, A., Caniatti, L., Rosaria Tola, M., Castellazzi, M., . . . Granieri, E. (2010). Altered miRNA expression in T regulatory cells in course of multiple sclerosis. Journal of Neuroimmunology, 226(1-2), 165-171. http://doi.org/10.1016/j.jneuroim.2010.06.009
Dinarello, C. A. (2000). Proinflammatory cytokines. Chest, 118(2), 503-8. http://doi.org/10.1177/0149206309356804
Furer, V., Greenberg, J. D., Attur, M., Abramson, S. B., & Pillinger, M. H. (2010). The role of microRNA in rheumatoid arthritis and other autoimmune diseases. Clinical Immunology. http://doi.org/10.1016/j.clim.2010.02.005
Goparaju, C. M., Blasberg, J. D., Volinia, S., Palatini, J., Ivanov, S., Donington, J. S., Pass, H. (2011). Onconase mediated NFKbeta downregulation in malignant pleural mesothelioma. Oncogene, 30(24), 2767-2777. http://doi.org/10.1038/onc.2010.643
Liang, D., & Shen, N. (2012). MicroRNA involvement in lupus: the beginning of a new tale. Current Opinion in Rheumatology, 24(5), 489-498. http://doi.org/10.1097/BOR.Ob013e3283563363
Lofgren, S. E., Frostegard, J., Truedsson, L., Pons-Estel, B. A., D'Alfonso, S., Witte, T., . . . Alarc6n-Riquelme, M. E. (2012). Genetic association of miRNA-146a with systemic lupus erythematosus in Europeans through decreased expression of the gene. Genes and Immunity, 13(3), 268-74. http://doi.org/10.1038/gene.2011.84
Mills, K. H. G. (2011). TLR-dependent T cell activation m autoimmunity. Nature Reviews Immunology, 11(12), 807-22. http://doi.org/10.1038/nri3095
Ohl, K., & Tenbrock, K. (2011). Inflammatory cytokines in systemic lupus erythematosus. Journal of Biomedicine & Biotechnology, 2011, 432595. http://doi.org/10.1155/2011/432595
Ortega, F. J., Moreno-Navarrete, J. M., Pardo, G., Sabater, M., Hummel, M., Ferrer, A., Fernndez-Real, J. M. (2010). MiRNA expression profile of human subcutaneous adipose and during adipocyte differentiation. PLoS ONE, 5(2). http://doi.org/10.1371/journal.pone.0009022
Pauley, K. M., Cha, S., & Chan, E. K. L. (2009). MicroRNA in autoimmunity and autoimmune diseases. Journal of Autoimmunity, 32(3-4), 189-194. http://doi.org/10.1016/j.jaut.2009.02.012
Savarino, A., Boelaert, J. R., Cassone, A., Majori, G., Cauda, R., & Raoult, D. (2003). Effects of chloroquine on viral infections: an old drug against today's diseases? The Lancet. Infectious Diseases, 3(11), 722-7. http://doi.org/10.1016/S1473-3099(03)00806-5
Schneeberger, M., Altirriba, J., Garda, A., Esteban, Y., Castano, C., Garda-Lavandeira, M., Claret, M. (2013). Deletion of miRNA processing enzyme Dicer in POMC-expressing cells leads to pituitary dysfunction, neurodegeneration and development of obesity. Molecular Metabolism, 2(2), 74-85. http://doi.org/10.1016/j.molmet.2012.10.001
Shen, R., Li, J., Ye, D., Wang, Q., & Fei, J. (2016). Combination of onconase and dihydroartemisinin synergistically suppresses growth and angiogenesis of non-small-cell lung carcinoma and malignant mesothelioma. Acta Biochimica et Biophysica Sinica, 48(10), 894-901. http://doi.org/10.1093/abbs/gmw082
Smigielska-Czepiel, K., van den Berg, a, Jellema, P., van der Lei, R. J., Bijzet, J., Kluiver, J., . . . Kroesen, B.-J. (2014). Comprehensive analysis of miRNA expression in T-cell subsets of rheumatoid arthritis patients reveals defined signatures of naive and memory Tregs. Genes and Immunity, 15(2), 115-25. http://doi.org/10.1038/gene.2013.69
Thom, R., Moraes, A. S., Bombeiro, A. L., Farias, A. dos S., Francelin, C., da Costa, T. A., Verinaud, L. (2013). Chloroquine Treatment Enhances Regulatory T Cells and Reduces the Severity of Experimental Autoimmune Encephalomyelitis. PLoS ONE, 8(6). http://doi.org/10.1371/journal.pone.0065913
Wiesen, J. L., & Tomasi, T. B. (2009). Dicer is regulated by cellular stresses and interferons. Molecular Immunology, 46(6), 1222-1228. http://doi.org/10.1016/j.molimm.2008.11.012
Zhang, X.-M., Guo, L., Chi, M.-H., Sun, H.-M., & Chen, X.-W. (2015). Identification of active miRNA and transcription factor regulatory pathways in human obesity-related inflammation. BMC Bioinformatics, 16, 76. http://doi.org/10.1186/s12859-015-0512-5
Zhou, X., Jeker, L. T., Fife, B. T., Zhu, S., Anderson, M. S., McManus, M. T., & Bluestone, J. A. (2008). Selective miRNA disruption in T reg cells leads to uncontrolled autoimmunity. The Journal of Experimental Medicine, 205(9), 1983-91. http://doi.org/10.1084/jem.20080707 EXAMPLES

### Example 1

### Ranpirnase Efficacy In Vitro

The objective of this experiment was to compare the efficacy of ranpirnase and FDA approved drugs for reducing SARS-CoV-2 concentration in vitro.

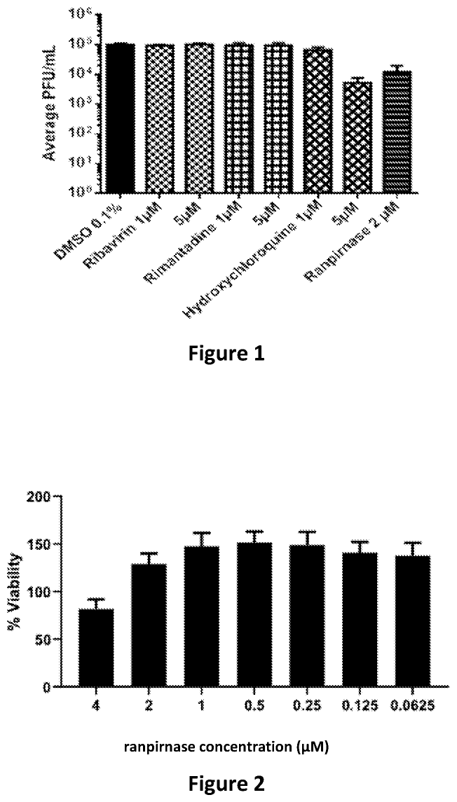

Ranpirnase was one of 2 drugs to reduce virus concentrations in the assay (FIG. 1) Similarly, UTHSC tested ranpirnase in their high-throughput screening (HTS) method for SARS-CoV-2 with the 50% effective dose (EC50) ˜6 μM.

Based on the in vitro activity against SARS-CoV-2, ranpirnase is expected to be active against SARS-CoV-2 in a hamster infection study, in which hamsters will be challenged with SARS-CoV-2 with analysis of lung infection on days 3, 5, and 7 to evaluate virus reduction in these tissues.

### Example 2

### Ranpirnase Safety

The objective of this experiment was to assess ranpirnase cytotoxicity in vitro.

Ranpirnase was added to Vero cells cultures, starting at a 4 μM and diluted 2-fold to 0.0625 μM. Cell viability was measured after 24 hours by CellTiter Glo.

Ranpirnase was not toxic at concentrations of 2 μM or less. Cells treated with 4 μM ranpirnase were 80% viable (FIG. 2).

### Example 3

### Ranpirnase Efficacy In Vitro

The objective of this experiment was to compare the efficacy of ranpirnase and 7G for reducing SARS-CoV-2 concentration in vitro.

Vero cells were incubated SARS-Cov-2 (MOI 0.1) DMEM medium at 37 C and 5% CO₂. 7G (100 μg/ml) or ranpirnase (2 μM) were added for 18 hours to the medium. Afterwards, 250 μl of supernatant was added to 750 μl of Trizol-LS for RNA extraction and qRT-PCR, and a part of the supernatant was also collected and plaqued for viral titer count.

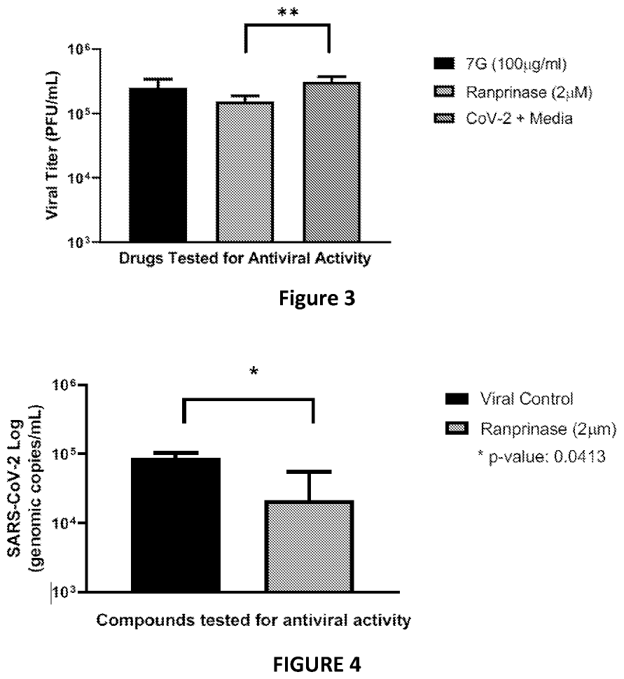

Ranpirnase, but not 7G, significantly reduced the viral titer count (FIG. 3). Further, ranpirnase reduced the number of genomic copies of SARS-CoV-2 (FIG. 4).

### Example 4

### Clinical Study of Ranpirnase for Treating Covid-19

The proposed clinical study is expected to present minimal risk to participants while contributing to development of a safe, efficacious COVID-19 therapeutic. The clinical research will be conducted in accordance with applicable International Conference on Harmonisation of Technical Requirements for Registration of Pharmaceuticals for Human Use (ICH) guidelines and FDA regulations and guidance. Ranpirnase will be tested in a human Phase II/III clinical trial to evaluate the technical feasibility of ranpirnase to reduce viral load and risk of an IL-6 cytokine storm in COVID-19 patients. Table 1 shows the Phase II/III protocol synopsis we are proposing to conduct. The study is designed for participation by adult (≥21 years old) COVID-19 patients. The clinical study will enroll up to 45 subjects with a 3:1 treatment:placebo (standard of care) ratio. All inclusion and exclusion criteria must be met for eligibility. Evaluation of this investigational therapeutic will include laboratory tests, medical history, physical assessment by clinicians, and subject self-assessment (if appropriate). The hypotheses are that ranpirnase is safe and reduces the viral burden in COVID-19 patients. Primary endpoints will be safety and efficacy. The assessment of product safety will include clinical observation and monitoring of hematological, and chemical parameters while efficacy assessment will evaluate virological measures.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1.-10. (canceled)

11. A composition comprising a ribonuclease and immunoglobulins, fragments thereof, antibodies, or combinations thereof, obtained from a plasma of a subject immune to a viral disease.

12. The composition of claim 11, wherein said ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

13. The composition of claim 11, wherein said ribonuclease comprises ranpirnase.

14. The composition of claim 11, wherein said viral disease comprises Covid-19, and said plasma is collected from:
a. a healthy subject or pool of subjects who have been previously exposed to SARS-CoV-2, naturally or by deliberate immunization, and who have IgG or IgM antibodies to SARS-CoV-2 virus in their plasma; or
b. a subject or pool of subjects where SARS-CoV-2 infection rate is high;
c. a subject or pool of subjects who have a history of SARS-CoV-2 infection in the past; or
d. a subject or pool of subjects who have antibodies as the result of deliberate immunization with SARS-CoV-2 or with antigens associated with SARS-CoV-2; or
e. any combination thereof.

15. The composition of claim 11, further comprising immune cells.

16. A composition comprising a ribonuclease and immune cells.

17. The composition of claim 16, wherein said ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

18. The composition of claim 16, wherein said ribonuclease comprises ranpirnase.

19. The composition of claim 15, wherein said immune cells are selected from a group comprising neutrophils, eosinophils (acidophiles), basophils, lymphocytes, monocytes, B cells, memory B cell, regulatory B cells (Breg), T cells, cytotoxic T cells, Helper T cells, Th1 cells, Th2 cells, Regulatory T cells (Treg), memory T cells, Natural Killer (NK) cells, monocytes, dendritic cells, macrophages, myeloid dendritic cells (mDC), plasmacytoid dendritic cell (pDC), or a combination thereof.

20. The composition of claim 15, wherein said immune cells are obtained from a donor, from a cell line, or from a subject immune to a viral disease.

21. The composition of claim 20, wherein said viral disease comprises Covid-19.

22. A method for treating or preventing a viral disease in a subject, said method comprising administering a composition comprising a ribonuclease.

23. The method of claim 22, wherein said ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

24. The method of claim 22, wherein said ribonuclease comprises ranpirnase.

25. The method of claim 22, wherein said viral disease is caused by a virus selected from a group comprising severe acute respiratory syndrome coronavirus 2 (SARS-CoV-2), an adenovirus, a herpesvirus, a papillomavirus, a polyomavirus, a poxvirus, an hepadnavirus, a parvovirus, an astrovirus, a calicivirus, a picornavirus, a coronavirus, a flavivirus, a togavirus, a hepevirus, a retrovirus, an orthomyxovirus, an arenavirus, a bunyavirus, a filovirus, a paramyxovirus, a rhabdovirus, a reovirus, Herpes simplex type 1, Herpes simplex type 2, Varicella-zoster virus, Epstein-Barr virus, Human cytomegalovirus, human herpesvirus type 8, human papillomavirus, BK virus, JC virus, smallpox, Hepatitis B virus, parvovirus B19, human astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, Banna virus, or any combination thereof.

26. The method of claim 22, wherein said viral disease is selected from a group comprising acute hepatitis, AIDS, aseptic meningitis, bronchiolitis, Burkitt's lymphoma, chickenpox, chronic hepatitis, common cold, congenital rubella, congenital varicella syndrome, congenital seizures in the newborn, croup, cystitis, cytomegalic inclusion disease, fatal encephalitis, gastroenteritis, German measles, gingivostomatitis, hepatic cirrhosis, hepatocellular carcinoma, herpes labialis, cold sores, herpes zoster, Hodgkin's lymphoma, hyperplastic epithelial lesions, warts, laryngeal papillomas, epidermodysplasia verruciformis, infectious mononucleosis, influenza, influenza-like syndrome, Kaposi sarcoma, keratoconjunctivitis, liver, lung and spleen diseases in the newborn, malignancies, cervical carcinoma, squamous cell carcinomas, measles, multicentric Castleman disease, mumps, myocarditis, nasopharyngeal carcinoma, pericarditis, pharyngitis, pharyngoconjunctival fever, pleurodynia, pneumonia, poliomyelitis, postinfectious encephalomyelitis, premature delivery, primary effusion lymphoma, rabies, Reye syndrome, severe bronchiolitis with pneumonia, skin vesicles, mucosal ulcers, tonsillitis, pharyngitis, or combination thereof.

27. The method of claim 22, wherein said viral disease comprises Covid-19, or wherein said viral disease is caused by severe acute respiratory syndrome coronavirus 2 (SARS-CoV-2).

28. The method of claim 22, further comprising immunoglobulins, fragments thereof, antibodies, or combinations thereof, obtained from a plasma of a subject immune to said viral disease.

29. The method of claim 28, wherein said viral disease comprises Covid-19, and said plasma is collected from:
a. a healthy subject or pool of subjects who have been previously exposed to SARS-CoV-2, naturally or by deliberate immunization, and who have IgG or IgM antibodies to SARS-CoV-2 virus in their plasma; or
b. a subject or pool of subjects where SARS-CoV-2 infection rate is high; or
c. a subject or pool of subjects who have a history of SARS-CoV-2 infection in the past; or
d. a subject or pool of subjects who have antibodies as the result of deliberate immunization with SARS-CoV-2 or with antigens associated with SARS-CoV-2; or
e. any combination thereof.

30. The method of claim 22, further comprising immune cells.

31. The method of claim 22, further comprising quinine, chloroquine, hydroxychloroquine, or analogues or derivatives thereof.


SEQUENCE LISTING (5 sequences)

SEQ ID NO: 1 - protein, 104 aa, Rana pipiens
EDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 2 - protein, 104 aa, Artificial Sequence
  ′805 variant of ranpirnase
EDWLTFQKKHVTNTRDVDCNNIMSTNLFHCKDKNTFIYSRPEPVKAICKGIIASKNVLTTSEFYLSDCNVTSRPCKYKLKKSTNKFCVTCENQAPVHFVGVGRC

SEQ ID NO: 3 - protein, 114 aa, Rana pipiens
KPKEDREWEKFKTKHITSQSVADFNCNRTMNDPAYTPDGQCKPVNTFIHSTTGPVKEICRRATGRVNKSSTQQFTLTTCKNPIRCKYSQSNTTNFICITCRDNYPVHFVKTGKC

SEQ ID NO: 4 - protein, 114 aa, Artificial Sequence
  rAmphirnase 2
KPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 5 - protein, 104 aa, Rana pipiens
EDWLTFQKKHITNTRDVDCDNIMSSNLFHCKDKNTFIYSRPEPVKAICKGIIASKNVLTTSEFYLSDCNVTSRPCKYKLKKSTNKFCVTCENQAPVHFVGVGSC